(19) United States
(12) Patent Application Publication
(10) Pub. No.: US 2004/0132966 A1
(43) Pub. Date: Jul. 8, 2004

(54) Backbone anchored thioester and selenoester generators

(76) Inventor: Leslie Philip Miranda, San Francisco, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/623,118

(22) Filed: Jul. 18, 2003

Related U.S. Application Data
(60) Provisional application No. 60/437,508, filed on Dec. 30, 2002.

(51) Int. Cl. C12N 9/16; C07K 14/00

(52) U.S. Cl. 530/324; 435/196; 558/233; 558/250

(57) ABSTRACT

Thioester and selenoester generators, precursors thereof, thioester and selenoester compounds produced therefrom, and related methods for their production are provided. The subject thioester and selenoester generators include an amino acid synthon having an N-terminal group joined to a C-terminal group through an organic backbone comprising one or more carbons. The organic backbone contains a backbone nitrogen, anchored to a support through a nucleophile-stable linker that lacks reactive functional groups. The organic backbone may include a target molecule of interest, such as an amino acid, peptide, polypeptide or other organic compound of interest, and/or the N- and/or C-termini can be elaborated using a variety of synthesis approaches to provide a target molecule of interest. The compounds and methods find a wide variety of uses, including use in thioester- or selenoester-based chemical ligation techniques.

### CROSS-REFERENCE TO RELATED APPLICATION

This application claims the benefit of U.S. provisional application serial No. 60/437,508, filed Dec. 30, 2002, which application is incorporated herein by reference in its entirety.

### BACKGROUND OF THE INVENTION

Thioesters and selenoesters represent an important class of molecules that readily react with nucleophiles. Thioesters are particularly useful for conjugation and chemoselective ligation reactions. Chemical ligation involves the chemoselective covalent linkage of a first chemical component to a second chemical component. Unique, mutually reactive functional groups present on the first and second components can be used to render the ligation reaction chemoselective. For example, thioesters are commonly used to direct the chemoselective chemical ligation of peptides and polypeptides. Several different thioester-mediated chemistries have been utilized for this purpose, such as native chemical ligation (Dawson, et al., Science (1994) 266:776-779; Kent, et al., WO 96/34878; Kent, et al., WO 98/28434).

Unfortunately, conventional preparation and use of peptide and other thioesters (Hojo, et al., Pept. Chem. (1992), Volume Date 1991, 29th pp.115-20;

Canne, et al. Tetrahed. Letters (1995) 36:1217-20; Hackeng, et al., Proc. Natl.

Acad. Sci., USA. (1999) 96:10068-73) have been limited to non-nucleophilic synthetic strategies. For example, when attempting to make thioester-activated peptides using N-α-9-fluorenylmethyloxycarbonyl (“Fmoc”)-based synthesis, the unwanted destruction of the thioester moiety by nucleophiles such as piperidine or piperidine-generated hydroxide ions during synthesis of the peptide will occur.

This is a significant problem, since the preferred reagent employed to remove Nα-Fmoc groups in each cycle of Fmoc-based organic synthesis contains piperidine. Piperidine, like other strongly basic or nucleophilic compounds (hereinafter “nucleophiles,”) destroys the thioester component of the peptide, rendering it useless for subsequent thioester-mediated reactions.

Several attempts have been made to address this problem. In one of the more promising approaches, Botti et al. (WO 02/18417) have reported on the application of nucleophile-stable carboxyester thiols or orthothioloester compounds for generating thioester and selenoester compounds. However, other efforts have met with limited success. For instance, Clippingdale et al. (J. Peptide Sci. (2000) 6:225-234) have used a non-nucleophilic base to remove Nα-Fmoc groups of peptides made using Fmoc-based Solid-Phase Peptide Synthesis (“SPPS”). This method has several problems, including generation of unwanted deletions, side-products, and requirement for backbone protection strategies. Other groups, including, Bertozzi et al. (J. Amer. Chem. Soc. (1999) 121:11684-11689) and Pessi et al. (Journal of the American Chemical Society; 1999; 121:11369-11374.), have reported adapting Fmoc SPPS in combination with a ‘Kenner’ safety-catch linker, which is stable to nucleophiles until the linker has been alkylated, to produce a fully protected peptide-thioester in solution. A drawback of this technique is the poor solubility properties of protected peptides in solution, as well as side reactions inherent to the method, such as the formation of unwanted alkylated byproducts when the linker is alkylated to render it labile, and thus it is impractical for many applications.

In addition, Barany, et al. (J. Org. Chem. (1999) 64(24):8761-8769) have reported on a Fmoc-SPPS method employing a backbone amide linker (“BAL”) to generate peptide thioesters on-resin. Among other problems, the BAL method is prone to diketopiperazine formation in the first few peptide extension cycles, reducing yields and its general application. Ishi et al. (Biosci. Biotechnol. Biochem. (2002) 66(2):225-232) have reported on the use of Fmoc-SPPS to generate Fmoc protected glycopeptide thioesters. As noted above, removal of Fmoc protecting groups is incompatible with thioesters, limiting the utility of this approach. Moreover, beyond a requirement for a serine or threonine anchored to a silyl ether linker based resin, the Ishi et al. method generates thioester products that are fully or substantially protected when released from the resin into solution. As noted above, such protected products exhibit poor solubility in solution, particularly in aqueous-based solutions. Similar frustration has been experienced in nucleophilic-based synthesis schemes for molecules other than peptides, such as small organic molecules.

Accordingly, there is a need for a universal and robust system for producing thioester- and selenoester-generating compositions compatible with organic or aqueous reaction conditions for use in various organic synthesis strategies, and conjugation and chemoselective ligation reactions that employ thioester- or selenoester-mediated reactions. The present invention satisfies these needs, as well as others, and generally overcomes deficiencies found in the background art.

### SUMMARY OF THE INVENTION

Thioester and selenoester generators, precursors thereof, thioester and selenoester compounds produced therefrom, and related methods for their production are provided. The subject thioester and selenoester generators include an amino acid synthon having an N-terminal group joined to a C-terminal group through an organic backbone that includes one or more carbons. The organic backbone contains a backbone heteroatom, e.g., nitrogen, anchored to a support through a nucleophile-stable linker. The organic backbone may include a target molecule of interest, such as an amino acid, peptide, polypeptide or other organic compound of interest, and/or the N- and/or C-termini can be elaborated using a variety of synthesis approaches to provide a target molecule of interest. The compounds and methods find use in a wide variety of applications, including use in thioester- or selenoester-based chemical ligation techniques.

In one representative embodiment, the subject thioester or selenoester generators include an amino acid synthon having an N-terminal group joined to a C-terminal group through an organic backbone that includes three or more amino acid residues having side chains lacking reactive functional groups, where the N-terminal group includes a first amino acid residue having a backbone nitrogen anchored to a support through a nucleophile-stable linker, the C-terminal group includes a second amino acid residue having a backbone carbonyl of an ester chosen from a thioester and a selenoester, and the first and second amino acid residues are separated by one or more additional amino acid residues.

Representative methods of preparing the generators of the above representative embodiment are also provided, where these methods include: (a) providing a precursor compound that includes an amino acid synthon having an N-terminal group joined to a C-terminal group through an organic backbone having three or more amino acid residues having side chains lacking reactive functional groups, where the N-terminal group includes a first amino acid residue having a backbone nitrogen anchored to a support through a nucleophile-stable linker and lacks reactive functional groups, the C-terminal group includes a second amino acid residue having a free backbone carboxyl, and the first and second amino acid residues are separated by one or more additional amino acid residues; and (b) converting the backbone carboxyl to a thioester or selenoester.

In another embodiment, a method of preparing a thioester or selenoester is disclosed, where the method includes: (a) providing a precursor composition that includes an amino acid synthon having an N-terminal group joined to a C-terminal group through an organic backbone lacking reactive functional groups, where the N-terminal group includes an unprotected backbone nitrogen anchored to a support through a nucleophile-stable linker, and said C-terminal group includes a backbone carboxyl protected with a carboxyl protecting group that is removable under conditions orthogonal to said nucleophile-stable linker; (b) coupling a peptide to said unprotected backbone nitrogen, where the peptide is preferably a dipeptide, more preferably a tripeptide or higher peptide, and includes a C-terminal group having an activated carboxyester and an N-terminal group having an amino group protected with an amino protecting group removable under conditions orthogonal to said carboxyl protecting group; (c) optionally, selectively removing the amino protecting group from the product of step (b) to generate an unprotected amino group, and producing an elongated product having one or more amino acids or peptides that extend from, and are covalently joined to said unprotected amino group, with the proviso that the elongated product is lacking reactive functional groups; (d) selectively removing the carboxyl protecting group from the product of step (b) or (c) to generate a free carboxyl group; and (e) converting the free carboxyl group to a thioester or selenoester to produce the thioester or selenoester.

Also provided are thioester or selenoester generator precursors compounds. In one representative embodiment, the thioester or selenoester generator precursors include an amino acid synthon having an N-terminal group joined to a C-terminal group through an organic backbone that lacks reactive functional groups, where the N-terminal group includes a backbone amino group anchored to a support through a nucleophile-stable linker and is protected with an amino protecting group, and the C-terminal group includes a backbone carboxyl protected with a carboxyl protecting group that is removable under conditions orthogonal to the backbone nitrogen protecting group.

The subject precursor compounds find use in the production of thioester or selenoester generators. One representative embodiment of such methods includes: (a) providing a thioester or selenoester generator precursor that includes an amino acid synthon having an N-terminal group joined to a C-terminal group through an organic backbone, where the N-terminal group includes a backbone heteroatom anchored to a support through a nucleophile-stable linker and an N-terminal amino protecting group, and the C-terminal group includes a backbone carboxyl protected with a carboxyl protecting group that is removable under conditions orthogonal to the amino protecting group; and (b) converting the protected backbone carboxyl moiety of the precursor to a thioester or selenoester to produce the thioester or selenoester generator.

In certain representative embodiments, the thioester or selenoester generators are sterically hindered thioester or selenoester generators, which include an amino acid synthon having an N-terminal group joined to a C-terminal group through an organic backbone, where the N-terminal group includes a heteroatom anchored to a support through a nucleophile-stable linker, and the C-terminal group includes a moiety chosen from a sterically hindered thioester or selenoester.

Representative methods of preparing the sterically inhibited generators of the above representative embodiment are also provided, where these methods include: (a) providing a precursor composition that includes an amino acid synthon having an N-terminal group joined to a C-terminal group through an organic backbone that lacks reactive functional groups, where the N-terminal group includes a backbone heteroatom anchored to a support through a nucleophile-stable linker, and the C-terminal group includes a free carboxyl; and (b) converting the free carboxyl of the precursor composition to a sterically hindered thioester or selenoester.

The thioester and selenoester generating compounds, the resulting thioester and selenoester compounds themselves, and the related methods greatly expand the capabilities of solid-phase synthesis schemes that employ or benefit from the use of thioesters or selenoesters, particularly for synthesis of target molecules by nucleophilic schemes such as solid-phase Fmoc-based peptide synthesis. The invention allows for the introduction of a variety of thioester and selenoester functionalities onto a target molecule of interest, particularly peptides and polypeptides. The invention may be employed in a wide range of thioester and selenoester mediated ligation reactions for production of peptides, polypeptides and other organic molecules capable of being constructed using ligation schemes employing thioesters and/or selenoesters. These and other objects and advantages of the invention will be apparent from the detailed description below.

### BRIEF DESCRIPTION OF THE DRAWINGS

The invention will be more fully understood by reference to the following drawings, which are for illustrative purposes only.

FIG. 1 is a general reaction scheme illustrating the synthesis of thioester and selenoester generators and thioester and selenoester peptides in accordance with an embodiment of the invention.

FIG. 2 is another general reaction scheme illustrating the synthesis of thioester and selenoester generators and thioester and selenoester peptides in accordance with an embodiment of the invention.

FIG. 3 is a specific reaction scheme illustrating an exemplary synthesis of thioester and selenoester generators and thioester and selenoester peptides in accordance with the invention.

FIG. 4 is another specific reaction scheme illustrating an exemplary synthesis of thioester and selenoester generators and thioester and selenoester peptides in accordance with the invention.

### DETAILED DESCRIPTION OF THE INVENTION

Disclosed herein are thioester- and selenoester-generators, thioester and selenoester compounds, and related methods for their production and use. The compounds and methods have wide applicability in organic synthesis for the generation of activated thioester and selenoesters. The subject compounds are particularly useful in peptide and polypeptide synthesis techniques that employ thioester and/or selenoester-mediated ligation, including native chemical ligation. The invention allows generation of activated thioesters and selenoesters from precursors that are prepared under strong nucleophilic conditions such as those occurring in Fmoc-(N^{α}-9-fluorenylmethyloxycarbonyl)-based peptide synthesis. The compounds of the invention support complex multi-step ligation or conjugation schemes.

The invention is described primarily in terms of use with Fmoc-compatible synthesis, including Fmoc-based solid-phase peptide and polypeptide synthesis (SPPS). Those skilled in the art will recognize, however, that the invention may be used for preparation of a variety of compounds having nucleophile-sensitive functionalities using various nucleophile-labile protecting group schemes. Moreover, those skilled in the art will recognize that the thioester and selenoester generators and related methods of the invention may be applied in tert-butyloxycarbonyl-(Boc) compatible synthesis, including Boc-based SPPS, as well as combinations of Fmoc- and Boc-compatible synthesis. Additional embodiments include 2-(4-nitrophenylsulfonyl)ethoxycarbonyl (Nsc.Bzi), allyloxycarbonyl (Alloc), and other protection schemes compatible with SPPS. The invention is also described primarily in terms of peptide synthesis involving chain extension from an Na terminus. Those skilled in the art will recognize that peptide synthesis involving extension from the C-terminus may also be carried out using the invention. Thus, it should be understood that the invention is not limited to the particular embodiments described below, as variations of these embodiments may be made and still fall within the scope of the appended claims. It should also be understood that the terminology employed is for the purpose of describing particular embodiments, and is not intended to be limiting. Instead, the scope of the present invention will be established by the appended claims. Any definitions herein are provided for reason of clarity, and should not be considered as limiting. The technical and scientific terms used herein are intended to have the same meaning as commonly understood by one of ordinary skill in the art to which the invention pertains.

Thioester and Selenoester Generators

The thioester and selenoester generators of the invention include, in general terms, an amino acid synthon having an N-terminal group joined to a C-terminal group through an organic backbone that includes one or more carbons. The organic backbone includes a backbone heteroatom, e.g., nitrogen, anchored to a support through a nucleophile-stable linker that lacks (i.e., is lacking) reactive functional groups. The organic backbone may include a target molecule of interest, such as an amino acid, peptide, polypeptide, or other organic compound of interest, and/or the N- and/or C-termini can be elaborated using a variety of synthesis approaches to include a target molecule of interest. The linker may also include a variety of linkers cleavable under non-nucleophilic conditions, such as linkers cleavable by strong acid, reduction, displacement reagents, light, and the like, and may include a target molecule of interest, or components of a target molecule, and can be of variable lengths.

In certain embodiments, the thioester- or selenoester-generators of the invention bear an N-terminal group having a moiety selected from: (i) a functional group protected with a nucleophile-labile protecting group, (ii) a functional group protected with a nucleophile-stable protecting group, (iii) an unprotected functional group, or (iv) an unprotected group that is substantially unreactive under conditions employed for generating the thioester- and selenoester-generators of the invention. A preferred N-terminal group comprises a moiety selected from a free amine, an amine protected with a nucleophile-stable amine protecting group, and an unprotected group lacking a reactive functionality, such as an unreactive alkyl or aryl capping moiety that may be linear, branched, substituted or unsubstituted.

In certain embodiments, the thioester- or selenoester-generators of the invention possess a C-terminal group having a moiety selected from: (i) a carboxyl protected with a carboxyl protecting group removable under conditions orthogonal to the N-terminal nucleophile-stable protecting group and linker, or (ii) a thioester or selenoester. The thioester- or selenoester-generators may comprise sterically hindered or non-hindered thioester or selenoester moieties.

The thioester- or selenoester-generators of the invention may be provided with such N- and C-terminal groups in various combinations, depending on the intended end use. As described above, the thioester and selenoester generators comprise an amino acid synthon having an N-terminal group joined to a C-terminal group through an organic backbone that includes one or more carbons, where the organic backbone lacks reactive functional groups and includes a heteroatom, e.g., nitrogen, anchored to a support through a nucleophile-stable linker. In one embodiment, the N-terminal group includes an unprotected or protected N-terminal group, with the proviso that the N-terminal protecting group is removable under non-nucleophilic conditions, and the C-terminal group comprises a moiety chosen from a thioester or selenoester. In another embodiment, the N-terminal group includes an unprotected or protected N-terminal group, and the C-terminal group includes a moiety chosen from a sterically hindered thioester or sterically hindered selenoester.

By “amino acid synthon” is intended a structural unit within a molecule, the structural unit comprising at least one amino acid or amino acid residue having an N-terminus comprising or extending from the alpha nitrogen of the amino acid or amino acid residue, a C-terminus comprising or extending from the alpha carbonyl of the amino acid or amino acid residue, and an organic backbone that joins the N- and C-termini and is substituted or unsubstituted with one or more side chains, where the structural unit can be formed and/or assembled by known or conceivable synthetic operations. In one embodiment, a thioester or selenoester generator according to the present invention includes an amino acid synthon having an N-terminal group joined to a C-terminal group through an organic backbone having side chains lacking reactive functional groups, where the N- and C-terminal groups each includes an amino acid residue, and are separated by at least one additional amino acid residue.

Examples of amino acid synthons are unprotected and partially or fully protected amino acids and peptides having a modified or unmodified alpha amino terminus (N-terminus) and/or a modified or unmodified alpha carbonyl terminus (C-terminus). This includes unactivated and activated esters thereof, as well as salts thereof, such as trifluoroacetic acid (TFA) salts. It also includes variable forms thereof in which the pendant N- and/or C-termini comprise terminal groups other than an alpha amino or carbonyl moiety, such as other amino acid non-functional and functional groups, one or more protecting groups, halogens, azides, conjugates, organic moieties other than an amino acid, a target molecule of interest, or components thereof, depending on the intended end use.

The term “amino acid” means any of the 20 genetically encodable amino acids, non-encoded amino acids, and analogs and derivatives thereof, including α-amino acids, β-amino acids, γ-amino acids, and other compounds having at least one N-terminal amino functionality and at least one C-terminal carboxyl (or carbonyl) functionality thereon. L- and D-forms of the chiral amino acids are also contemplated. The terms “peptide”, “polypeptide” and “protein”, which may be used interchangeably herein, refer to an oligomeric or polymeric form of amino acids, which can include coded and non-coded amino acids, chemically or biochemically modified or derivatized amino acids, and polypeptides having modified peptide backbones.

In the context of an amino acid synthon, an “organic backbone” may include the alpha, beta and/or gamma carbons of a single amino acid residue, and other substituents, including additional backbone carbons and/or heteroatoms, as well as alpha amino groups of an amino acid or residue that are substituted or unsubstituted (amides included), alpha carbonyls that are substituted or unsubstituted (carboxyls, carboxyesters and amide bonds included), and may comprise an amino acid residue or peptide, as well as organic side chains. Representative organic side chains are those of amino acids. The organic backbone typically comprises part or most of a target molecule of interest.

By “lacking reactive functional groups” is intended a group or radical in which such reactive functional groups are entirely absent, as well as a group or radical that contains protected functional groups that would otherwise be reactive but for the presence of the protecting group(s).

Accordingly, the organic backbone may be fully protected, partially protected or unprotected depending on the intended end use. For example, the organic backbone may have one or more side chains bearing a functional group protected with a protecting group removable under conditions orthogonal to the N-terminal protecting group. This structure is particularly convenient where the organic backbone is constructed using Fmoc-compatible synthesis, and the N-terminal protecting group, if present, is removable under conditions orthogonal to Fmoc-removal. In this situation, the organic backbone may include a peptide chain containing amino acid residues bearing protected functional groups removable under conditions orthogonal to nucleophilic removal of an N-terminal Fmoc group during peptide elongation cycles, such as nucleophile-stable/acid-cleavable protecting groups, and where the last amino acid coupling includes an N-terminal protecting group cleavable under conditions different from Fmoc or side-chain protecting group removal, such as catalytic hydrogenation conditions (e.g., an Alloc group).

In other instances, the organic backbone may contain one or more side chains bearing a functional group protected with a protecting group that is removable under the same conditions as the N-terminal protecting group. For example, both the N-terminal protecting group and the side chains can be protected with nucleophile-stable, acid-cleavable protecting groups, so that the side chains and N-terminal group can be deprotected in one step. Particularly useful nucleophile-stable protecting groups cleavable under acidic conditions include the tert-butyl (tBu), tert-butyloxycarbonyl (Boc), and trityl (Trt) groups.

Alternatively, the organic backbone may contain one or more side chain functional groups that are substantially non-reactive to conditions used for generating or manipulating a target molecule attached to the support, and/or side chains that would otherwise be reactive but are protected with protecting groups that are orthogonal to such generating or manipulating conditions.

The term “orthogonal” as used herein with respect to protecting groups, linkers and other groups means that the specific group or linker is removable or cleavable under conditions that do not result in removal or cleavage of an “orthogonal” group or linker. Thus, for example, where the linker is nucleophile-stable and the N-terminal group bears a nucleophile-labile protecting group, cleavage of the linker is “orthogonal” to removal of the nucleophile-labile protecting group, and vice versa.

For instance, when the organic backbone is made to contain cysteine amino acid residues, the side chain thiol can be protected with an acetamidomethyl (Acm) or Picolyl group, which are stable to basic conditions (e.g., typical conditions for Fmoc-compatible cycles during primary target synthesis) or acidic conditions (e.g., typical Boc-compatible cycles and/or conditions for final deprotection and cleavage of an elongated thioester or selenoester target molecule from the support). Protecting groups like Acm- and Picolyl also are removable under conditions orthogonal to carbonyl protecting groups such as Allyl or ODMab. The same orthogonal protection strategy can be employed with other side chains, for example, side chains bearing a primary amine protected with an Alloc group. Where the organic backbone contains side chain functional groups that are substantially unreactive, protection of those groups is typically not required. Examples of side chain groups that are substantially unreactive include alcohols, and other such groups can be selected depending on the conditions employed.

The above stratagems also can be exploited with respect to the nucleophile-stable linker. For instance, the N-terminal protecting group and the nucleophile-stable linker can be provided in a combination where they are cleavable under orthogonal conditions. Alternatively, the N-terminal protecting group and the nucleophile-stable linker can be selected so that they are both cleavable under the same conditions. Many such linkers are known, and can be selected for this purpose, including those described in further detail herein. Preferred linkers stable to nucleophiles such as piperidine are cleavable under conditions such as acid or light. These include a wide range of linkers, with the most preferred linkers being compatible with Fmoc-based, Boc-based, Alloc-based, and/or peptide synthesis. The linkers may employ multi-detachable components, including dual linker systems, as well as contain spacers or other divalent linker elements.

Linkers usable with the invention include, for example, PAL (5-(4′-aminomethyl-3′,5′-dimethoxyphenoxy)valeric acid, XAL (5-(9-aminoxanthen-2-oxy)valeric acid), 4-(alpha-aminobenzyl)phenoxyacetic acid, 4-(alpha-amino-4′-methoxybenzyl)phenoxybutyric acid, p-alkoxybenzyl (PAB) linkers, photolabile σ-nitrobenzyl ester linkers, 4-(alpha-amino-4′-methoxybenzyl)-2-methylphenoxyacetic acid, 2-hydroxyethylsulfonylacetic acid, 2-(4-carboxyphenylsulfonyl)ethanol, (5-(4′-aminomethyl-3′,5′-dimethoxyphenoxy)valeric acid) linkers, WANG hydroxymethyl phenoxy-based linkers, RINK trialkoxybenzydrol and trialkoxybenzhydramine linkers, and Sieber aminoxanthenyl linkers. PAM, SCAL, and other linker systems may also be used. These linker systems are cleavable under well known acidolysis conditions (typically trifluoroacetic acid (TFA) or hydrogen fluoride (HF)), UV photolysis (λ=350 nm) conditions, or catalytic hydrogenation conditions. Several of the above linker systems are commercially available as pre-formed on resin and glass supports.

The support of the thioester and selenoester generators of the invention comprises a solid phase, matrix or surface compatible with organic synthesis strategies. Preferred supports are those compatible with peptide synthesis. A variety of such supports are well known, and can be employed, including those described in further detail herein. Examples include supports or resins comprising cross-linked polymers, such as divinylbezene cross-linked polystyrene polymers, or other organic polymers that find use for solid-phase organic or peptide synthesis. Controlled porous glass (CPG) supports are another example. In general, the most preferred supports are stable and possess good swelling characteristics in many organic solvents.

With respect to the side chain of the organic backbone, the side chain is preferably an amino acid side chain. Examples of preferred amino acid side chains include those of glycine, alanine, valine, leucine, isoleucine, serine, threonine, cysteine, methionine, aspartic acid, asparagines, glutamic acid, glutamine, arginine, lysine, histidine, phenylalanine, tyrosine, tryptophan, and proline. Of course, substantial diversity can be provided for side chains beyond the typical amino acid side chains mentioned above for particular uses consistent with the invention disclosed herein.

As described above, the thioester and selenoester generators of the invention may have a modified or unmodified alpha amino terminus (N-terminus) and/or a modified or unmodified alpha carbonyl terminus (C-terminus). In a preferred embodiment, the thioester and selenoester generators of the invention have an N-terminal group that comprises an amino acid. Any amino acid can be used. Preferably, the amino acid is capable of supporting chemical ligation. Chemical ligation involves the selective covalent linkage of a first chemical component to a second chemical component. Orthogonally reactive functional groups present on the first and second components can be used to render the ligation reaction chemoselective. For example, chemical ligation of peptides and polypeptides involves the chemoselective reaction of peptide or polypeptide segments bearing compatible, mutually reactive C-terminal and N-terminal amino acids. Several different chemistries have been utilized for this purpose, examples of which include native chemical ligation (Dawson, et al., Science (1994) 266:776-779; Kent, et al., WO 96/34878; Kent et al., U.S. Pat. No. 6,184,344), extended general chemical ligation (Kent, et al., WO 98/28434; and Kent et al., U.S. Pat. No. 6,307,018); extended native chemical ligation (Botti et al., WO 02/20557); oxime-forming chemical ligation (Rose, et al., J. Amer. Chem. Soc. (1994) 116:30-33), thioester forming ligation (Schnblzer, et al., Science (1992) 256:221-225), thioether forming ligation (Englebretsen, et al., Tet. Letts. (1995) 36(48):8871-8874), hydrazone forming ligation (Gaertner, et al., Bioconj. Chem. (1994) 5(4):333-338), and thiazolidine forming ligation and oxazolidine forming ligation (Zhang, et al., Proc. Natl. Acad. Sci. (1998) 95(16):9184-9189; Tam, et al., WO 95/00846) or by other methods (Yan, L. Z. and Dawson, P. E., “Synthesis of Peptides and Proteins without Cysteine Residues by Native Chemical Ligation Combined with Desulfurization,” J. Am. Chem. Soc. 2001, 123, 526-533; Gieselnan et al., Org. Lett. 2001 3(9):1331-1334; Saxon, E. et al., “Traceless” Staudinger Ligation for the Chemoselective Synthesis of Amide Bonds. Org. Lett. 2000, 2, 2141-2143). Preferred chemical ligation methods employ amide-forming chemical ligation, such as native chemical ligation and extended native chemical ligation.

By “capable of supporting chemical ligation” is intended a moiety that is in a form that can be directly employed in a chemical ligation reaction, or can be converted to a moiety for use in a chemical ligation reaction. In many situations, moieties capable of supporting chemical ligation will be in a form that must be converted for a ligation reaction to proceed. For instance, when a thioester or selenoester generator of the invention is employed for making a target molecule bearing an N-terminal amino acid capable of supporting chemical ligation in combination with a C-terminal thioester or selenoester, the N-terminal amino acid is typically protected to avoid intramolecular cyclization or undesired intermolecular condensation with itself. In this way, such a target molecule can be used for a thioester or selenoester-mediated chemical ligation reaction, such as native or extended native chemical ligation, followed by removal of the N-terminal protection for subsequent native or extended native chemical ligation reaction cycles (e.g., sequential native or extended native chemical ligation). In some instances, however, intramolecular cyclization may be desired, which is particularly useful for making cyclic products, such as cyclic peptides. N-terminal amino acids, such as serines, that are capable of being converted to bear an aldehyde moiety by mild oxidation or reductive alkylation is another example, which find particular use in Schiff-base mediated chemical ligation reactions. In other chemical ligation reactions, the N-terminal amino acid can be provided in a ready-to-use chemical ligation form, such as when the N-terminal amino acid bears an azide, halogen, or aminooxy group for other chemical ligation reactions.

Where the N-terminal group comprises an amino acid capable of supporting native or extended native chemical ligation, the amino acid comprises a side chain bearing an atom selected from sulfur and selenium. Examples of amino acids suitable for use in native chemical ligation comprise an alpha-carbon side chain bearing a sulfur or selenium atom, such as cysteine, homocysteine, selenocysteine, homoselenocysteine, and protected forms thereof. Examples of amino acids suitable for use in extended native chemical ligation comprise an alpha-nitrogen side chain bearing a sulfur or selenium atom, which include the alpha-nitrogen substituted 2 or 3 carbon chain alkyl or aryl thiol and selenol auxiliaries, and protected forms thereof as described in Botti et al., WO 02/20557. As can be appreciated, an N-terminal amino acid capable of supporting native or extended native chemical ligation can be protected using a protecting group for the alpha-nitrogen, the side chain sulfur or selenium, or a combination of both, including cyclic protection strategies employing an N-terminal thioproline or extended native chemical ligation alpha-nitrogen substituted auxiliary. The thioester and selenoester generators of the invention preferably employ an amino acid bearing a side chain sulfur or selenium group that is protected.

As described above, the C-terminal group of the thioester and selenoester generators of the invention includes a thioester and selenoester. This includes any group compatible with the thioester or selenoester group, including, but not limited to, aryl, benzyl, and alkyl groups that may be linear, branched, substituted or unsubstituted, which includes amino acid, peptide and other organic thioester or selenoester moieties. Preferred examples include 3-carboxy-4-nitrophenyl thioesters, benzyl thioesters and selenoesters, mercaptopropionyl thioesters and selenoesters, and mercaptopropionic acid leucine thioesters and selenoesters (See, e.g., Dawson et al., Science (1994) 266:776-779; Canne et al. Tetrahedron Lett. (1995) 36:1217-1220; Kent, et al., WO 96/34878; Kent, et al., WO 98/28434; Ingenito et al., J. Am. Chem. Soc (1999) 121(49):11369-11374; and Hackeng et al., Proc. NatI. Acad. Sci. U.S.A. (1999) 96:10068-10073).

In one embodiment, the C-terminal group includes a sterically hindered thioester or selenoester having the formula J—CH(R1)—C(O)—X—R2, where J is a residue of the organic backbone; R1 is any compatible side chain group; X is sulfur or selenium; and R2 is any thioester or selenoester compatible group; and where one or both of R1 and R2 is a group that sterically hinders the thioester or selenoester moiety —C(O)—X—. In a preferred embodiment, one of R₁ and R₂ is selected from a branching group having the formula —C(R₄)(R₅)(R₆), where R₄, R₅ and R₆ each individually are selected from hydrogen and linear, branched, substituted and unsubstituted alkyl, aryl, heteroaryl, and benzyl groups, with the proviso that two or more of R₄, R₅ and R₆ are selected from linear, branched, substituted and unsubstituted alkyl, aryl, heteroaryl, and benzyl groups. The C-terminal group bearing either a sterically hindered or non-hindered thioester or selenoester preferably comprises an amino acid.

By way of example, a thioester and selenoester generator that includes an amino acid synthon having an N-terminal group joined to a C-terminal group through an organic backbone having one or more carbons, is described by the formula:

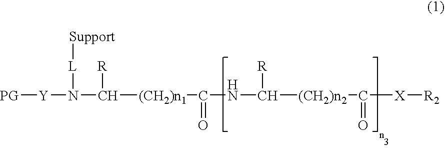

wherein PG is a nucleophile-stable protecting group that may be present or absent; Y is a target molecule of interest that may be present or absent and is preferably lacking reactive functional groups; “Support” is a solid phase, matrix, or surface; L is a nucleophile-stable linker; each R individually is hydrogen or an organic side-chain lacking reactive functional groups; n₁ and n₂ each are from 0 to 2; n₃ is from 0 to 20 and preferably from 2 to 20; X is oxygen, sulfur or selenium; and R₂ is a protecting group removable under conditions orthogonal to PG when X is oxygen, and is any group compatible with thioesters or selenoesters when X is sulfur or selenium.

In compounds of the structure (1), PG is a nucleophile-stable protecting group that can be removed under conditions orthogonal to, or the same as the nucleophile-stable linker L. Alternatively, PG can be absent. The presence or absence of PG and the particular PG employed is chosen based on the N-terminal group of Y. For instance, where the N-terminal group of Y comprises an amino group, such as the alpha amino group of an amino acid, exemplary nucleophile-stable amino protecting groups usable for PG include, by way of example, Boc and benzyloxycarbonyl (Cbz) protecting groups, which respectively are removable under mild acidic and mild catalytic hydrogenation conditions. As described above, the N-terminal group may include a protected or unprotected amino acid. Where the target molecule of interest is designed as an intermediate for subsequent chemical ligation reactions, a preferred N-terminal amino acid is capable of supporting chemical ligation. Examples of N-terminal amino acids capable of supporting chemical ligation include cysteine residues bearing an N-alpha amino protected with PG, or an N-alpha amino protected with PG that is substituted with an auxiliary side chain bearing a thiol or selenol for general or extended native chemical ligation. For N-terminal ligation groups, the thiols, selenols or other nucleophiles are preferably protected with nucleophile-stable protecting groups such as Acm or benzyl derivatives. Where Y includes an N-terminal group that is substantially non-reactive, such as a linear, branched, substituted or unsubstituted aliphatic or other capping group, then PG can be absent, for example, where further elaboration of the support bound target molecule is desired. Alternatively, a reactive group may be present on the N-terminal group, but is generally chosen so as not to react with the C-terminal thioester or selenoester, except where thioester- or selenoester-mediated intramolecular cyclization is desired.

The group Y may include any molecule of interest including, for example, an amino acid, peptide, polypeptide, nucleic acid, lipid, carbohydrate, combinations thereof, and the like. Preferred Y groups are peptides.

The linker L may include any cleavable group capable of anchoring the organic backbone nitrogen atom to the support material that is stable to nucleophilic conditions. As linker L is stable to nucleophilic conditions, it is cleavable under conditions orthogonal to the conditions for removal of nucleophile-labile protecting groups, such as Fmoc groups.

The use of linker groups in solid-phase synthesis is well known, and various linker groups L are usable with the invention. The linker L may be bifunctional, and may serve as a spacer with a cleavable functional group on one end, and a group such as a carboxyl group at the other end that can be activated to allow coupling to a functionalized support material. The linker can be a preformed linker or may be prepared on a support material. Suitable linkers L include, for example, PAL, XAL, PAM, RINK, SCAL, and Sieber-based linker systems as described above. The aforementioned linkers are non-silyl-based linkers or are otherwise lacking a silyl group. Linkers that include a silyl ether group are less preferred, but may be employed in certain embodiments where silyl ether linkages are desired.

Linker L is covalently anchored to a support as described further below. Suitable supports may include, for example, matrixes, surfaces, resins or other solid phase or support that is compatible with peptide synthesis or other synthetic schemes associated with the target molecule Y. The support may include a functionalized glass, an organic polymer, or other material. Suitable solid supports are described in, for example, “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998, and elsewhere (See, e.g., G. B. Fields et al., Synthetic Peptides: A User's Guide, 1990, 77-183, G.A. Grant, Ed., W. H. Freeman and Co., New York; NovaBiochem Catalog, 2000; “Synthetic Peptides, A User's Guide,” G.A. Grant, Ed., W. H. Freeman & Company, New York, N.Y., 1992; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Fmoc Solid Phase Peptide Synthesis, A Practical Approach,” W. C. Chan and P. D. White, Eds., Oxford Press, 2000).

The term “organic group” and “organic radical,” as used herein, means a hydrocarbon group that is classified as an aliphatic group, cyclic group, aromatic group, functionalized derivatives thereof and/or various combination thereof. The term “aliphatic group” means a saturated or unsaturated linear or branched hydrocarbon group and encompasses alkyl, alkenyl, and alkynyl groups, for example. The term “alkyl group” means a saturated linear or branched hydrocarbon group including, for example, methyl, ethyl, isopropyl, t-butyl, heptyl, dodecyl, octadecyl, amyl, 2-ethylhexyl, and the like. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group. The term “heterocyclic group” means a closed ring hydrocarbon in which one or more of the atoms in the ring is an element other than carbon (e.g., nitrogen, oxygen, sulfur, etc.). The organic groups may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc.

The group R may comprise hydrogen or any organic side-chain lacking reactive functional groups. In this regard, R may comprise an amino acid side chain, with the amino acid glycine corresponding to the case where R comprises hydrogen. Where R is a side chain associated with an amino acid that has a reactive functionality on the side chain such as glutamic acid, a suitable protecting group or groups may be used so that R is lacking a reactive functional group. As such, R may include a side chain of an amino acid selected from aspartic acid, glutamic acid, glutamine, lysine, serine, threonine, arginine, cysteine, histidine, tryptophan, tyrosine, and asparagine. Alternatively, R may bear a functional group that is otherwise substantially unreactive under the conditions employed in a given synthesis step of interest. Such substantially unreactive functional groups can include primary or secondary alcohols, or aminooxy or ketone moieties, for example, when cycles of activation, acylation and deprotection procedures are employed in peptide synthesis. It will be appreciated that protecting groups for R, as well as each R can vary independently with each component bearing such R group in the organic backbone.

As discussed above, the group R₂ is a protecting group removable under conditions orthogonal to PG when X is oxygen. When X is sulfur or selenium, however, R₂ may include any group that is compatible with a thioester or selenoester, such as alkyl, aryl, and benzyl groups, including phenyl, t-butyl, and ethyl carboxy alkylate groups. Such R₂ groups may also include amino acids and peptides, and other organics. Various activated thioesters and selenoesters are known, and suitable divalent radicals associated with such thioester and selenoesters are employable, and may be used with the invention.

Compounds of the structure (1) represent a variety of intermediates usable for thioester and selenoester generation. As described above, where Y is absent and where n3 is zero, the structure (1) corresponds to a single amino acid bound to linker L. Where nl is zero, the amino acid is an alpha-amino acid, and where n1 is 1 or 2, the amino acid correspondingly comprises a P-amino acid or a γ-amino acid. Where n3 is 1, 2, 3, or higher and Y is absent, the compound (1) corresponds respectively to a dipeptide, tripeptide, and tetrapeptide or higher peptide, which may comprise alpha, beta and gamma amino acids respectively where n2 is 0, 1, or 2. In certain embodiments, n3 is from 0 to 20, with 2 to 10, 2 to 5, 2 to 3, and 2 being the most preferred in this order. Where Y is present, the compound (1) may include a longer peptide, a peptide-polymer conjugate, or other peptide or polypeptide compound as described above.

In another embodiment, and by way of example, a sterically hindered thioester and selenoester generator includes an amino acid synthon having an N-terminal group joined to a C-terminal group through an organic backbone having one or more carbons, as described by the formula:

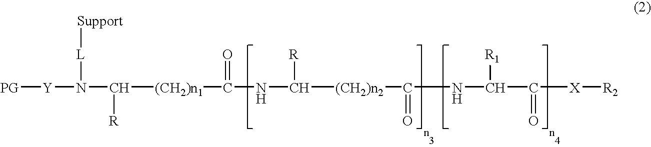

where PG is a protecting group that may be present or absent; Y is a target molecule of interest that may be present or absent and is preferably lacking functional reactive groups; L is a nucleophile-stable linker; Support is a solid phase, matrix, or surface; each R and R₁, individually, is any side chain group and may be the same or different and are lacking functional reactive groups; n₁ and n₂, each individually, is 0, 1, or 2; n₃ is 0 to 20; n₄ is 0 to 10, preferably 0 or 1; X is sulfur or selenium; and R₂ is any thioester or selenoester compatible group; and wherein one or more of R, R₁, and R₂ is a group that sterically hinders the thioester or selenoester moiety —C(O)—X—.

In the compounds of the structure (2), the Y and L groups are the same as described above for compounds of the structure (1). In the structure (2), protecting group PG may be any protecting group, including nucleophile-stable and nucleophile-labile protecting groups, and may be present or absent. In structure (2), an additional C-terminal alpha amino acid may optionally be present with a group R₁, which may comprise hydrogen or any organic side chain group. In structure (2), n₃ preferably is a number ranging from 0 to 20, 0 to 15, with 0 to 10, 0 to 5, 0 to 3, 0 to 2, and 0 to 1 being the most preferred in this order, i.e., 0 to 1 being most preferred. In the compounds of structure (2), at least one of the groups R, R₁, and R₂ is a group that sterically hinders the —C(O)— X-moiety. The terms “sterically hindering” and “sterically hindered” as used herein refers to a group or groups that prevent or help prevent hydrolysis or self-induced aminolysis associated with the —C(O)—X— moiety. The sterically hindering group R₁ and/or R₂ additionally aids in preventing racemization of the carbon bound to the R1 group where n₄ is 1. Where n₂ and n₄ are 0 and n₃ is greater than 0, the sterically hindering R and/or R₂ group helps to prevent unwanted side reactions associated with the carbon bound to the R group.

Sterically hindering groups usable for R, R₁, and/or R₂ include, by way of example, branched alkane, cycloalkane, alkyl-substituted aryl, and heteroaryl groups, and combinations thereof. Such sterically hindering groups may comprise the formula —C(R₄)(R₅)(R₆), or as alternatively presented:

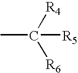

where R₄, R₅, and R₆ each individually comprise hydrogen, a linear, branched, cyclic substituted or unsubstituted alkyl, aryl, heteroaryl, or benzyl group, and at least two of R₄, R₅, and R₆ each individually comprise a linear, branched, cyclic substituted or unsubstituted alkyl, aryl, heteroaryl, or benzyl group. Other groups providing steric hindrance for the thioester or selenoester moiety may also be used for R, R₁, and/or R₂.

The use of the aforementioned protecting groups, linkers, solid phase supports, as well as specific protection and deprotection reaction conditions, linker cleavage conditions, use of scavengers, and other aspects of solid-phase peptide synthesis are well known and are also described in “Protecting Groups in Organic Synthesis,” 3rd Edition, T. W. Greene and P. G. M. Wuts, Eds., John Wiley & Sons, Inc., 1999; NovaBiochem Catalog, 2000; “Synthetic Peptides, A User's Guide,” G. A. Grant, Ed., W. H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994; “Fmoc Solid Phase Peptide Synthesis, A Practical Approach,” W. C. Chan and P. D. White, Eds., Oxford Press, 2000, G. B. Fields et al., Synthetic Peptides: A User's Guide, 1990, 77-183, and elsewhere.

Methodology for Synthesis of Thioester and Selenoester Generators

The thioester and selenoester generators of the invention can be prepared by providing a precursor composition having a free C-terminal carboxyl, followed by conversion of the free carboxyl to a thioester or selenoester to form the desired thioester or selenoester generator. In particular, the precursor composition includes an amino acid synthon having an N-terminal group joined to a C-terminal free carboxyl through an organic backbone that comprises a backbone heteroatom, e.g., nitrogen, anchored to a support through a nucleophile-stable linker. The organic backbone lacks reactive functional groups and the N-terminal group can be unprotected or protected, depending on the intended end use.

When a non-sterically hindered thioester or selenoester is desired, it is preferred that the N-terminal group and the C-terminal carboxyl each includes an amino acid residue, and are separated by one or more additional amino acid residues along the organic backbone. In addition, the N-terminal group is unprotected, or protected with a nucleophile-stable protecting group. Presence of a nucleophile-stable protecting group permits removal of the protecting group under non-nucleophilic conditions (i.e., in the presence of the formed thioester or selenoester), without destroying the thioester or selenoester moiety. When the N-terminal group is unprotected, it is preferred to be a group that is substantially non-reactive under conditions for carboxyl activation and coupling of a thioester or selenoester component. When a sterically hindered thioester or selenoester is desired, the N-terminal group may be protected or unprotected. In this situation, the protecting group can be nucleophile-stable or -labile. For an unprotected N-terminus, here again it is preferred that the N-terminus bears a group that is substantially non-reactive under conditions for carboxyl activation and coupling of a thioester or selenoester component.

Conversion of the free carboxylate of the precursor composition to the thioester or selenoester involves contacting an activated form of the free carboxyl with a compound selected from a thiol moiety, a selenol moiety, a preformed thioester, and a preformed selenoester. Activation of the free carboxyl can be carried out by any number of activating agents that are capable of forming a carboxyester. Preferred carboxyl activation techniques include in situ activation and/or the use of preformed activated amino acid derivatives such as commercially available pentafluorophenyl (OPfp) activated esters. Activating reagents capable of providing in situ generation of activated carboxyesters (OAct) include, by way of example, Obt (benzotriazoly carboxy ester) and OAt (azabenzotriazoly carboxy ester) activation reagents such as DIC/HOBt, HATU, PyBOP, PyAOP, TBTU, HBTU, and like activation systems. Other activation reagents, such as TFFH (acid fluoride activation), may also be used. Activation can be carried out in the presence of thiol moiety, a selenol moiety, a preformed thioester, and a preformed selenoester, or can be provided in a pre-activated form followed by the addition of the thiol moiety, a selenol moiety, a preformed thioester, and a preformed selenoester. An advantage of the former approach is a reduction in overall reaction time, which reduces potential for racemization or other unwanted side-reactions.

In a preferred embodiment, the compound bearing the thiol or selenol moiety used in the thioester or selenoester conversion process comprises the formula HS—R₂ or HSe—R₂. The R₂ group is as defined above, and may be any group compatible with thioesters or selenoesters. This includes linear, branched, substituted and unsubstituted alkyl, aryl, heteroaryl, and benzyl groups. For example, mercaptans and senenols, such as mercaptoproprionic acid, mercaptoproprionyl, thiophenol, selenophenol, selenolproprionic acid, and selenolproprionyl compounds can be used for this purpose.

Alternatively, a preformed thioester or selenoester compound may be used in the thioester or selenoester conversion process. These preformed thioester or selenoester compounds preferably comprise an amino acid or peptide. This includes preformed thioester or selenoester compounds of the formula H[NH—C(R₁)—C(O)]ₙ₅—S—R₂ ; and H[NH—C(R₁)—C(O)]ₙ₅—Se—R₂; where R₁ and R₂ are as defined above, and each individually are the same or different and are lacking reactive functional groups; where n₅ is from 1 to 5, with n₅ preferably being from 1 to 4, with 1 to 3, 1 to 2, and 1 being the most preferred in this order. For example, chemically synthesized thioester and selenoester amino acids and peptides can be made from the corresponding α-thioacids or α-selenoacids, which in turn, can be synthesized on a thioester- or selenoester resin or in solution, although the resin approach is preferred. The α-thioacids or selenoacids can be converted to the corresponding 3-carboxy-4-nitrophenyl thioesters or selenoesters, to the corresponding benzyl ester, or to any of a variety of alkyl thioesters or selenoesters. As another example, a trityl-associated mercaptoproprionic acid leucine thioester- or selenoester- generating resin can be utilized (Hackeng et al., supra). Thioester and selenoester synthesis also can be accomplished using a 3-carboxypropanesulfonamide safety-catch linker by activation with diazomethane or iodoacetonitrile followed by displacement with a suitable thiol or selenol (Ingenito et al., supra; Shin et al., J. Am. Chem. Soc. (1999) 121:11684-11689). Various other synthetic approaches for making preformed thio- or selenoesters may be employed as well (e.g., Beletskaya et al., Mendeleev Commun. (2000) 10(4):127-128; Kim et al., J. Chem. Soc., Chem. Commun. (1996) 1335; Dowd et al., J. Am. Chem. Soc. (1992) 114:7949; Wang et al., Synthetic Comm. (1999) 29(18):3107-3115; Lu et al., Synthetic Comm. (1999) 29(2):219-225; and Kozikowski et al., Tetrahedron (Symposium Series) (1985) 41:4821-4834).

The sterically hindered thioester and selenoester generators of the invention may be prepared by converting the free carboxyl of the precursor composition to a sterically hindered thioester or selenoester. This can be accomplished by coupling a compound comprising a sterically hindered thiol or selenol moiety to an activated form of the free carboxyl. In a preferred embodiment, the sterically hindered thiol or selenol moiety comprises the formula X—C(R₄)(R₅)(R₆), or as alternatively presented:

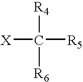

where X is a thiol or selenol; and R₄, R₅ and R₆ each individually are selected from the group consisting of hydrogen and linear, branched, substituted and unsubstituted alkyl, aryl, heteroaryl, and benzyl groups, with the proviso that two or more of R₄, R₅ and R6 are selected from the group consisting of linear, branched, substituted and unsubstituted alkyl, aryl, heteroaryl, and benzyl groups.

The sterically hindered thioester and selenoester generators also may be prepared using preformed sterically hindered thioester or selenoesters. This process involves converting the free carboxyl group to a sterically hindered thioester or selenoester by coupling a preformed amino acid or peptide having a sterically hindered thioester or selenoester to form an amide bond therein between. In this instance, the preformed amino acid or peptide thioester or selenoester comprises an unprotected N-terminal amine and a sterically hindered C-terminal thioester or sterically hindered selenoester.

Sterically hindered thioester and selenoester generators also may be prepared by converting a sterically hindered C-terminal carboxyl group to a thioester or selenoester. A sterically hindered C-terminal carboxyl group for this purpose comprises the formula:

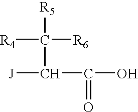

where J comprises a residue of the organic backbone; R₄, R₅ and R₆ each individually are any side chain lacking a reactive functional group and are selected from the group consisting of hydrogen and linear, branched, substituted and unsubstituted alkyl, aryl, heteroaryl, and benzyl groups, with the proviso that two or more of R₄, R₅ and R₆ are selected from the group consisting of linear, branched, substituted and unsubstituted alkyl, aryl, heteroaryl, and benzyl groups. Conversion of the sterically hindered C-terminal carboxylate to a sterically hindered thioester or selenoester may be carried out in combination with non-hindered thiols, selenols, preformed thioesters and preformed selenoesters, as well as sterically hindered versions thereof.

In a preferred embodiment, and by way of example, a preferred method for producing a thioester and selenoester generator comprising an amino acid synthon having an N-terminal group joined to a C-terminal group through an organic backbone having one or more carbons is carried out as follows. First, a precursor composition is provided having the formula:

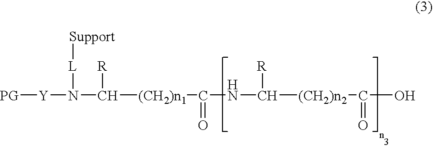

where PG, Y, L, Support, R, n₁, n₂, and n₃ are as defined above for structure (1). The free carboxyl of structure (3) is then converted to a thioester or selenoester to form a thioester or selenoester generator having the formula:

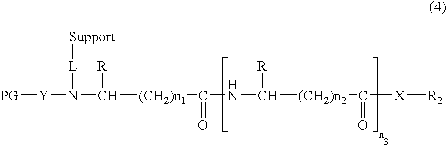

where X is sulfur or selenium; and R2 is as defined above for structure (1).

In another embodiment, and by way of example, a method for producing a sterically hindered thioester and selenoester generator that includes an amino acid synthon having an N-terminal group joined to a C-terminal group through an organic backbone having one or more carbons is carried out as follows. First, a precursor composition is provided having the formula:

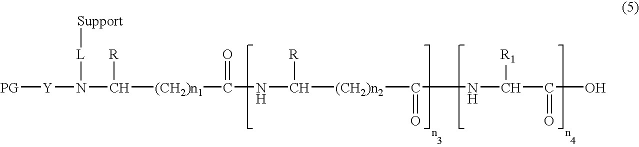

where PG, Y, L, Support, R, R₂, n₁, n₂, n₃ and n₄ are as defined above for structure (2). The free carboxyl of structure (5) is then converted to a sterically hindered thioester or selenoester to form a sterically hindered thioester or selenoester generator having the formula:

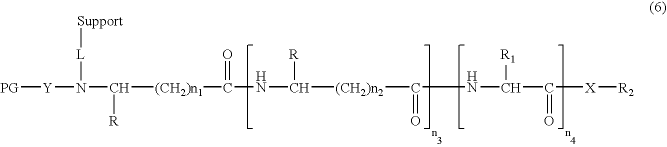

where X is sulfur or selenium; and R₁ and R₂ is as defined above for structure (2).

The activation of carboxyl groups as described above, as well as protection and deprotection and linker cleavage protocols, and solid-phase peptide synthesis generally are also described in “Protecting Groups in Organic Synthesis,” 3rd Edition, T. W. Greene and P. G. M. Wuts, Eds., John Wiley & Sons, Inc., 1999; NovaBiochem Catalog, 2000; “Synthetic Peptides, A User's Guide,” G. A. Grant, Ed., W. H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994; “Fmoc Solid Phase Peptide Synthesis, A Practical Approach,” W. C. Chan and P. D. White, Eds., Oxford Press, 2000, G. B. Fields et al., Synthetic Peptides: A User's Guide, 1990, 77-183, and elsewhere, as noted above.

Nucleophile-based Synthesis of Thioester and Selenoester Generators

The thioester and selenoester generators of the invention also can be prepared by a nucleophile-based synthesis scheme. This approach is particularly useful where nucleophiles are employed in the synthesis of a target molecule of interest, such as a peptide or polypeptide prepared by Fmoc- or Nsc-SPPS. The method may be employed to make sterically hindered and non-hindered thioesters and selenoesters. The method involves, in one embodiment, the following steps (a) through (e).

Step (a) First, a composition is provided that comprises an amino acid synthon having an N-terminal group joined to a C-terminal group through an organic backbone comprising one or more carbons. The N-terminal group of the composition comprises a reactive functional group protected with a nucleophile-labile protecting group, and the C-terminal group comprises a carboxyl protected with a carboxyl protecting group removable under conditions orthogonal to the nucleophile-labile protecting group. The organic backbone is lacking reactive functional groups and comprises a backbone heteroatom, e.g., nitrogen, anchored to a support through a nucleophile-stable linker cleavable under conditions orthogonal to the carboxyl protecting group. Thus, the linker and the nucleophile-labile and carboxyl protecting group pairing employed in Step (a) are removable under orthogonal conditions, and the carboxyl protecting group is stable to the conditions employed for removal of the N-terminal nucleophile-labile protecting group. The organic backbone may also comprise a target molecule of interest, or portion thereof.

As described above, the preferred support is compatible with solid-phase organic synthesis (SPOS) or solid-phase peptide synthesis (SPPS). The preferred nucleophile-stable linkers are removable under acidic conditions as provided by trifluoroacetic acid (TFA) or hydrogen fluoride (HF), under catalytic conditions in the presence of H2, or by other mechanism such as light (e.g., UV photolysis). The amino acid synthon will preferably be composed of an amino acid having a backbone heteroatom, e.g., nitrogen, anchored to the support through the linker, and may be provided in the initial composition as a single amino acid residue, peptide, or an organic composition containing an amino acid component, peptide, or residue thereof. As also noted above, the organic backbone is lacking reactive functional groups. In most instances, protecting groups, if present on the organic backbone, are preferably selected so as to be removable under the same conditions as the linker. However, protecting groups can be selected that provide an additional level of orthogonality when site-specific modifications to the organic backbone are desired during or after synthesis.

For the C-terminal group, exemplary carboxyl protecting groups removable under conditions orthogonal to the nucleophile-labile protecting group are Allyl and ODmab. Allyl groups are stable to nucleophiles, yet are removable by palladium-catalyzed hydrogenation. ODmab groups can be removed with hydrazine, which is a very strong nucleophile, but are stable to typical conditions employed for removal of most other nucleophile-labile protecting groups, such as N-terminal amino protecting groups Fmoc and 2-(4-nitrophenylsulfonyl)ethoxycarbonyl (Nsc Bzi). For instance, Fmoc and Nsc groups are readily removed by piperidine, which is a much weaker nucleophile compared to hydrazine. This difference in stability provides the appropriate level of orthogonality.

Depending on the N-terminal functional group used, various nucleophile-labile protecting groups may be employed, such as nucleophile-labile amino protecting groups where the N-terminal functional group is an amine, e.g., Fmoc and Nsc. As can be appreciated, other nucleophile-labile and carboxyl protecting groups having compatible orthogonality as described may also be employed in Step(a).

By way of example, preferred compositions employable in Step (a) comprise the formula:

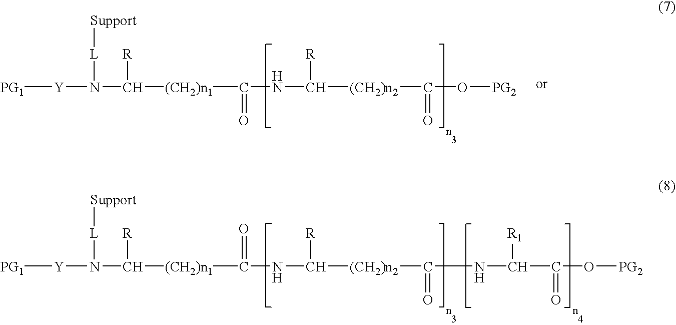

Referring to structures (7) and (8), PG₁ is a nucleophile-labile protecting group; Y is a target molecule of interest that may be present or absent; L is a nucleophile-stable linker; each R and Rp individually is hydrogen or any organic side-chain lacking reactive functional groups; ni and n2feach are from 0 to 2; n₃ is from 0 to 20; n₄ is 0 to 1; and PG2 is any protecting group that is removable under conditions orthogonal to removal of PG, and cleavage of L. Y. L, Support, Rl R₁, and nb₁, n₂, n₃ and n₄are as described above for the structure (2), with the proviso that Y. L, Support, R, R₁, are compatible with nucleophile-based SPOS and/or SPPS.

In order to prevent the formati on of diketopiperazine in the first coupling or elaborative step, n₃, n₄, and Y are such that the initial amino acid synthon of structures (7) or (8) includes at least three amino acid residues. Alternatively, where the initial amino acid synthon includes fewer than three amino acid residues, a dipeptide, tripeptide or higher peptide is coupled during the first coupling cycle to effectively prevent diketopiperazine formation, as discussed in further detail in step (c) below.

The protecting group PGl may comprise any of a variety of nucleophile-labile protecting groups. As noted above, the particular protecting group PG1 may be selected based on the particular molecule of interest or target molecule, compatibility with other protecting groups or functionalities that will be present during synthesis, or other considerations. The protecting group PG₂ may comprise any group capable of protecting a carboxyl group and is orthogonal to the nucleophile-labile protecting group PG₁ and the nucleophile-stable linker L, as discussed above. Exemplary protecting groups PG₂ and PG₁ fitting these criteria include allyl and ODmab groups for the C-terminal carboxyl protection, Fmoc and Nsc when the N-terminal group is an amine, and where a suitable linker would be one cleavable under acidic conditions.

Compositions of the structures (7) and (8) are easily extensible using conventional Fmoc-based or Nsc-based solid-phase organic or peptide synthesis (i.e., SPOS or SPPS) techniques, and provide for a backbone-based anchoring during synthesis for elaborating a target molecule of interest Y. For instance, structures (7) and (8) can be employed in a variety of nucleophile-based chain elongation synthesis schemes involving repeated cycles of nucleophilic deprotection and coupling with incoming compounds bearing a reactive moiety and PG₁, as illustrated below for structure (7).

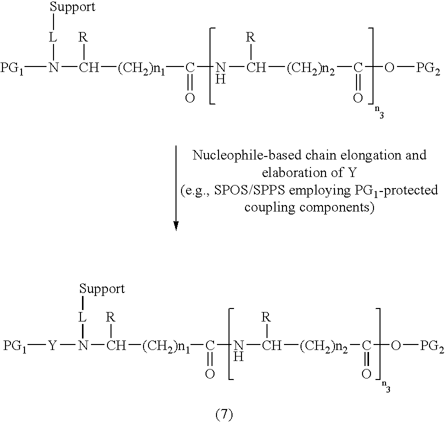

Step (b) From a composition provided in Step (a), the nucleophile-labile protecting group is then selectively removed under nucleophilic conditions to form an N-terminal group comprising a first reactive functional group. For instance, where PG, is a nucleophile-labile amino protecting group, and the pendant N-terminal group of Y is an amine, PG1 can be Fmoc or Nsc, and removal, thereof, can be carried out under basic conditions that do not remove PG2.

By way of example, compositions of certain embodiments generated in Step (b) comprise the formula:

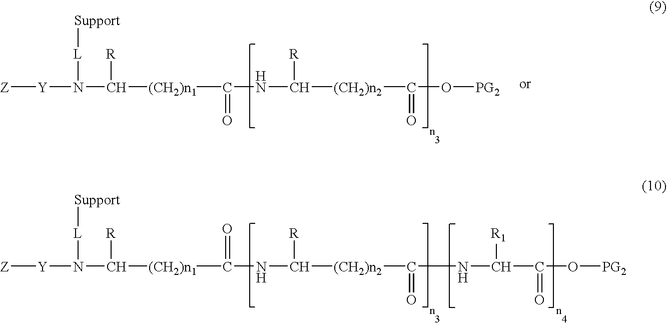

where Y, L, Support, R, R₁, n₁, n₂, n₃ and n₄ are as defined above for structure (2), with the proviso that Y, L, Support, R, R₁ are compatible with nucleophile-based SPOS and/or SPPS; and Z comprises a reactive functional group of interest.

Step (c) Following removal of the nucleophile-labile protecting group in Step (b), the deprotected N-terminal reactive functional group of the product of Step (b) is coupled to a compound of interest. The compound of interest bears a single reactive moiety capable of forming a covalent bond with the N-terminal reactive functional group. Various compounds can be employed in this step, depending on the intended end use, to generate an elongated product having the compound of interest on the N-terminal group.

In one embodiment of Step(c), hereinafter referred to as Step (c-i), an unprotected compound may be used for the coupling in Step(c). As such, the unprotected compounds will bear a single reactive moiety capable of forming a covalent bond with the N-terminal reactive functional group. Preferred unprotected compounds for Step (c-i) are those that are substantially unreactive in the presence of carboxyl activation agents and thiols or selenols, i.e., conditions employed for nucleophile-based synthesis of the thioester or selenoester. Examples of suitable unprotected compounds for Step (c-i) include mono-functionalized compounds that are missing other functional reactive groups, or have additional functional groups that are substantially unreactive under conditions employed for nucleophile-based synthesis of the thioester or selenoester, such as mono-functionalized amino acids, peptides, and other organics in which all but the single reactive moiety are capped, monofunctionalized conjugates, dyes, fluorescent labels, or tracers, radioactive elements, metal chelators, and the like, as well as mono-functionalized alkyls, aryls, benzyls, polymers, and the like. Unprotected compounds for Step (c-i) having additional functional groups that are substantially unreactive under conditions employed for nucleophile-based synthesis of the thioester or selenoester, include, for example, alcohols and ketones. Unprotected compounds for Step (c-i) may also include bi-functional moieties (e.g., diacids and diamines), or moieties that generate a new reactive functional group following coupling (e.g., amino and acid anhydrides). For the bifunctional moieties, the newly generated functionally group will typically require capping or protection prior to subsequent thioesterification or selenoesterfication.

In another embodiment of Step (c), hereinafter referred to as Step (c-ii), an amino-protected compound can be used for the coupling. In this situation, the amino-protected compound of Step (c-ii) comprises a single reactive moiety capable of forming a covalent bond with the N-terminal reactive functional group, and bears an amino group that is protected with a nucleophile-stable amino protecting group removable under conditions orthogonal to removal of the carboxyl protecting group. Thus, such amine-protected compounds of Step (c-ii) lack reactive functional groups other than a single reactive moiety that forms the covalent bond with the N-terminal reactive functional group of the product of Step (b). Examples of suitable amino-protected compounds of Step (c-ii) include amino acids, peptides, and other organics possessing an amino functionality. Monoamines, diamines, or higher amines are other examples.

In yet another embodiment of Step (c), hereinafter referred to as Step (c-iii), the coupling may be carried out with a protected compound having a single reactive moiety that forms a covalent bond with the N-terminal reactive functional group of the product of Step (b), and one or more additional reactive functional groups protected with a protecting group that is removable under conditions orthogonal to removal of the carboxyl protecting group. Protected compounds for Step (c-iii) are particularly useful for forming sterically hindered thioesters or selenoesters. Preferred examples of protected compounds for Step (c-iii) include amino acids and peptides, and other organic compounds having more than one reactive functional group, and include the amine-protected compounds for Step (c-iii).

By way of example, preferred compositions generated in Step (c) comprise the formula:

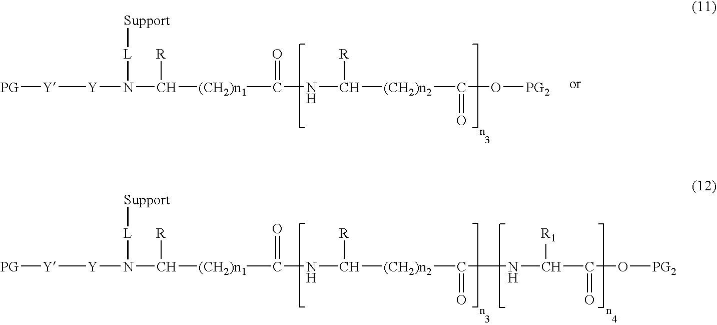

where Y, L, Support, R, R₂, n₁, n₂, n₃ and n₄ are as defined above for structure (2), with the proviso that Y. L, Support, R, R₂ are compatible with nucleophile-based SPOS and/or SPPS. Referring to structure (11), Y′ is a compound of interest lacking reactive functional groups; and PG may be present or absent, with the proviso that when present, PG is a nucleophile-stable amino protecting group removable under conditions orthogonal to PG₂ and Y′ bears an N-terminal amino group that is protected by PG. Referring to structure (12), Y′ is a compound of interest lacking reactive functional groups; and PG may be present or absent, with the proviso that PG is removable under conditions orthogonal to PG₂.

Referring to structures (11) and (12), when n₃ and n₄ are both 0 and Y is absent, it is preferred that the initial compound of interest Y′ is a compound unable to form a diketopiperazine, e.g., a dipeptide, tripeptide or higher peptide. Once the initial compound of interest Y′ is coupled to the N-terminal group, additional amino acids, peptides or other compounds of interest may be coupled to the amino acid synthon. Alternatively, the formation of diketopiperazine can also be effectively avoided when n₃, n₄ and Y are such that the initial amino acid synthon includes at least three backbone amino acid residues.

Step (d) Following the coupling of a compound of interest to the deprotected N-terminal reactive functional group in Step (c), the C-terminal carboxyl protecting group of that product is selectively removed to generate a free carboxyl group. Conditions for removing the carboxyl protecting group are chosen based on the protecting group employed. For instance, where an allyl group is employed, palladium-catalyzed hydrogenation can be used, or where an ODmab group is employed, the appropriate hydrazine cocktail can be used.

By way of example, preferred compositions generated in Step (d) comprise the formula:

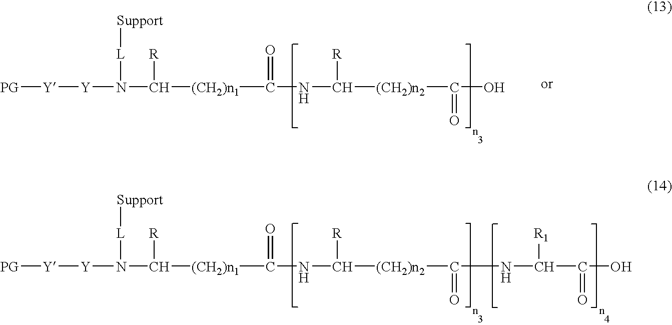

where PG, Y, L, Support, R, R1, n₁, n₂, n₃ and n₄ are as defined above for structure (2) and Y′ is as defined in structure (11).

Step (e) Following the selective removal of the C-terminal carboxyl protecting group, and generation of a free carboxyl group in Step (d), the free carboxyl group of the product of Step (d) is converted to a thioester or selenoester. The type of thioester or selenoester formed can vary depending on the compound of interest employed in the coupling step, and thus the compound present on the N-terminus of the product generated in Step (d).

In particular, it is preferable to covert the free carboxyl of the product of Step (d) to a sterically hindered thioester or selenoester when the product of Step (d) bears a protected compound from Step (c-iii) on its N-terminus, i.e., a protected compound having one or more reactive functional groups protected with a protecting group removable under conditions orthogonal to the carboxyl protecting group employed in Steps (a)-(e), regardless of the type of protecting group(s) present on the protected compound of interest. For instance, an exemplary protected compound from Step (c-iii) is any amino acid protected with any number of different protecting groups, including amino protecting groups removable under nucleophilic conditions, such as Fmoc or Nsc. In this situation, a sterically hindered thioester or selenoester moiety can provide some protection against nucleophilic cleavage if one desires to remove the nucleophile-labile protecting group in the presence of the thioester or selenoester, particularly where non-nucleophilic bases are employed. In most cases, however, a protected compound coupled in Step (c) will bear a nucleophile-stable protecting group.

Conversion of the free carboxyl group of a product of Step (d) that is formed with an unprotected compound of Step (c-i) or amine-protected compound of Step (c-iii) may be carried out to generate either sterically hindered or non-hindered thioesters or selenoesters.

As described above, a preformed thioester or selenoester, or compounds bearing a thiol or selenol moiety, may be coupled to an activated form of the free carboxyl of the product of Step (d) to convert, and thus generate the desired thioester or selenoester.

By way of example, preferred compositions generated in Step (e) comprise the formula:

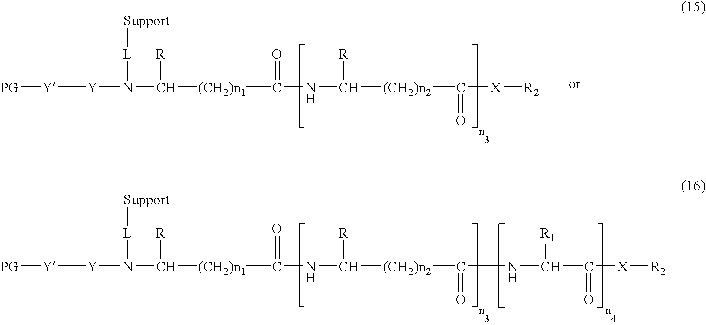

Referring to structure (15), Y. L, Support, R, n₁, n₂, and n₃ are as defined above for structure (1); Y′ is as defined in structure (11), PG may be present or absent and comprises a nucleophile-stable protecting group; and X is sulfur or selenium; and R₁ and R₂ are as defined above for structure (2). Referring to structure (16), Y. L, Support, R, n₁, n₂, n₃ and n₄ are as defined above for structure (2); Y′ is as defined for structure (12), PG may be present or absent; X is sulfur or selenium; and R₁ and R₂ are as defined above for structure (2).

At this stage, the support-bound thioester or selenoester product can be further elaborated or modified, for example, by on-support modifications to the organic backbone/target molecule of interest. In most instances any additional elongation or modifications are preferably those that do not damage the thioester or selenoester moieties. For example, where the N-terminal group bears a protecting group removable under non-nucleophilic conditions, it is possible to carry out one or more additional cycles of SPOS or SPPS using a non-nucleophilic synthesis scheme, e.g., Boc-SPPS. Coupling of additional reactive groups generally unstable to nucleophilic cycles of chain elongation, carboxyl activation, thioester or selenoester formation, can be performed at this stage. This is particularly useful when one desires to modify the N-terminus with a functional group such as an aldehyde, acid, conjugate group, or other group or structure. As another example, side chains of the organic backbone/target molecule of interest that were chosen to be orthogonal to reagents and conditions employed in Steps (a)-(e), and are removable under conditions orthogonal to the linker, can be removed and those side chains modified. It also may desirable to generate cyclic forms of the product of Step (e) while still bound to the support. This may be accomplished where the pendant N-terminal group bears, for example, a functional group reactive with thioesters or selenoester that is protected with a protecting group removable under conditions orthogonal to the linker and compatible with thioesters or selenoesters (e.g., an Acm-protected N-terminal Cysteine). Thus, once the N-terminal protecting group is removed, the support-bound material can form a cyclic product.

The organics, equipment, supports, amino acids and diversity components, linkers, and protecting groups finding use in the above nucleophile-based method can be obtained from a variety of commercial sources, prepared de novo, or a combination thereof. Moreover, the reagents and other materials employed for the method, as well as alternative components will be apparent to one of ordinary skill in the art (See, e.g., “Protecting Groups in Organic Synthesis,” 3rd Edition, T. W. Greene and P. G. M. Wuts, Eds., John Wiley & Sons Inc., 1999; NovaBiochem Catalog, 2000; “Synthetic Peptides, A User's Guide,” G. A. Grant, Ed., W. H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994, “Fmoc Solid Phase Peptide Synthesis, A Practical Approach,” W. C. Chan and P. D. White, Eds., Oxford Press, 2000, and elsewhere).

Methodology for Synthesis of Thioester and Selenoester Compounds

The thioester and selenoester generators of the invention, as described above, find particular use in the generation of thioester and selenoester compounds. The methods for generating thioester and selenoester compounds in accordance with the invention comprise, in general terms, providing a composition comprising an amino acid synthon having an N-terminal group joined to a C-terminal group through an organic backbone comprising one or more carbons, the organic backbone comprising a backbone heteroatom, e.g., a backbone nitrogen, anchored to a support through a nucleophile-stable linker and lacking reactive functional groups, the C-terminal group comprising a thioester or selenoester moiety, the N-terminal group comprising an unprotected or protected N-terminal group, with the proviso that the N-terminal protecting group is removable under non-nucleophilic conditions, and cleaving the linker under non-nucleophilic conditions to generate a thioester or selenoester compound free of the support. In certain embodiments, the freed thioester or selenoester compounds are fully or substantially unprotected and are soluble in aqueous solutions.

The above support-bound composition is carried out in generally the same manner as described above for the preparation or generation of thioester and selenoester generators. Cleavage of the linker to form the freed thioester or selenoester may be carried out under various conditions according to the nature of the linker used and the orthogonality of protecting groups present in the composition with respect to the linker. The linker may comprise PAL, XAL, PAB, PAM, SCAL, RINK, WANG, Sieber amide, and other linker systems as described above.

Where an N-terminal protecting group is present, cleavage of the linker may be carried out under conditions orthogonal to removal of the N-terminal protecting group, as well as orthogonal to any protecting groups for side chain groups associated with the amino acid synthon, such that the freed thioester or selenoester compound is fully protected. Such orthogonal conditions may comprise, for example, linker cleavage under acid conditions where the N-terminal protecting group is nucleophile labile. Linker cleavage may alternatively involve non-orthogonal conditions that also result in removal of the N-terminal protecting group and/or one or more amino acid side chain protecting groups that may be present on the organic backbone, such that the freed thioester or selenoester compound is partially protected or unprotected. Selection of various protecting groups and orthogonality of removal of protecting groups with respect to linker cleavage may be made based on desired synthetic schemes and solubility characteristics for the freed thioester or selenoester compounds.

The organic backbone may comprise a residue of an amino acid, peptide, polypeptide, or like moiety comprising alpha, beta and/or gamma amino acids, and may comprise one or more amino acid side groups which may be protected or unprotected depending upon side group functionality and desired use, as described above. The C-terminal and N-terminal groups may comprise protected or unprotected amino acids, and the N-terminal group may be capable of supporting chemical ligation to form an amide bond or other bond by various ligation techniques, including native chemical ligation and extended native chemical ligation as also described above. In this regard, the N-terminal group, in many embodiments, may comprise an amino acid with a protected or unprotected side chain functional group that is capable of participating in a chemical ligation reaction, such as thiol or selenol or other group containing a sulfur or selenium atom. The side chain functional group may be associated with a backbone carbon of an N-terminal amino acid or, in the case of extended chemical ligation, be associated with the alpha amine of an N-terminal amino acid.

The methods of generating thioester and selenoester compounds may comprise, more specifically, providing a composition of the formula:

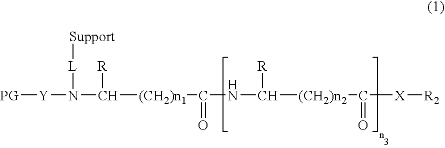

wherein PG, Y, L, Support, R, R₂, X, n₁, n₂, and n₃ are as described above for the structure (1).

Providing the above composition and cleaving of the linker may be carried out as described above, and PG, Y, L, R, R₂, X, n₁, n₂, n₃, and the Support are the same as related above in the description of the thioester and selenoester generators and related methodologies. The thioester or selenoester compound thus freed from the support may comprise the formula:

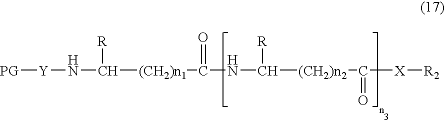

or, where PG is removable under the same conditions used for cleavage of linker L, the thioester or selenoester compound may comprise the formula:

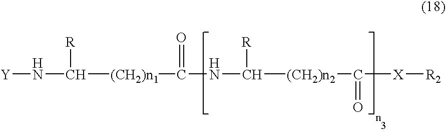

where Y, R, R₂, X, n₁, n₂, and n₃ are as described above for structure (1).

In certain embodiments, the methods may comprise:

(a) providing:

(i) a precursor compound having the formula:

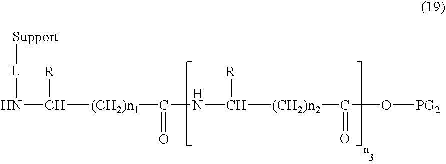

wherein:

L is a nucleophile-stable linker;

Support is chosen from a solid phase, matrix, or surface;

R is hydrogen or any organic side chain lacking reactive functional groups;

n₁ and n₂ each are from 0 to 2;

n₃ is from 0 to 20; and

PG₂ is a carboxyl protecting group; and

(ii) a peptide of the formula:

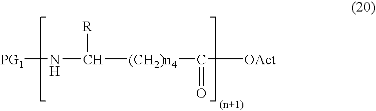

wherein:

PG₁ is an amino protecting group removable under conditions orthogonal to PG₂;

n4is 0 to 2;

n is 1 to 20; and

OAct is an activated ester;

(b) coupling the peptide (formula 20) to the unprotected amino group of the precursor compound (formula 19) to generate a composition having the formula:

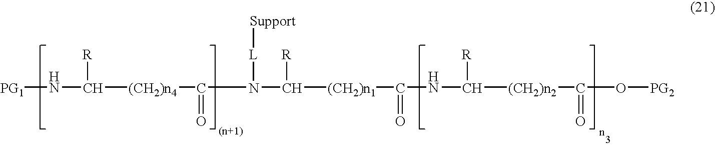

(c) optionally, selectively removing the amino protecting group PG, from the product of step (b) to generate an N-terminal group comprising an unprotected amino group, and forming an elongated product having the formula:

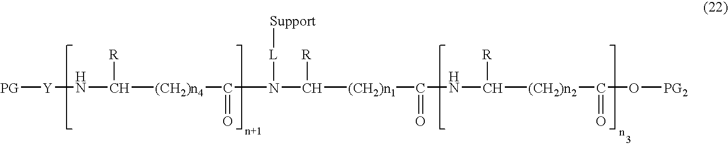

where:

Y is a target molecule of interest and is lacking reactive functional groups; and

PG is a protecting group that may be present or absent and is removable under conditions orthogonal to said carboxyl protecting group PG₂;

(d) selectively removing the carboxyl protecting group PG₂ from the product of step (b) or (c) to generate a composition having the formula:

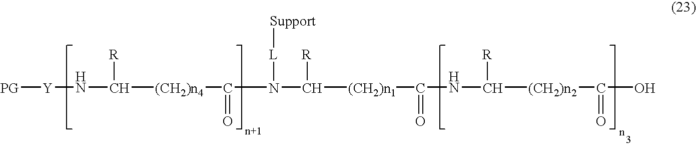

where:

PG and Y may be individually present or absent; and

(e) converting the product of step (d) to a thioester or selenoester to generate a thioester or selenoester generator of the formula:

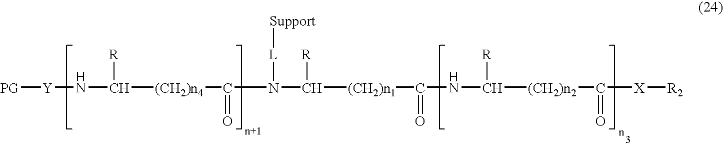

where:

PG and Y may be individually present or absent;

X is sulfur or selenium; and

R₂ is any group compatible with thioesters or selenoesters.

The invention also provides methods for generating sterically hindered thioester and selenoester compounds, comprising: providing a composition comprising an amino acid synthon having an N-terminal group joined to a C-terminal group through an organic backbone comprising one or more carbons, the organic backbone comprising a backbone heteroatom, e.g., nitrogen, anchored to a support through a nucleophile-stable linker and lacking reactive functional groups, the N-terminal group comprising an unprotected or protected N-terminal group, the C-terminal group comprising a sterically hindered thioester or selenoester moiety; and cleaving the linker under non-nucleophilic conditions to generate a sterically hindered thioester or selenoester compound free of the support.

The sterically hindered thioester or selenoester compounds freed from the support may be soluble in aqueous solution, and may be protected, partially protected or unprotected as described above. The organic backbone may be associated with a target molecule and may comprise an amino acid, peptide or polypeptide with one or more side chains bearing protected or unprotected functional groups, and the C-terminal and N-terminal groups may themselves comprise protected or unprotected amino acid groups as also described above. The N-terminal group may be capable of supporting chemical ligation, and may comprise an amino acid with a protected or unprotected side chain functionality capable of participating in native chemical ligation, extended chemical ligation or other ligation technique to form an amide bond.

The sterically hindered thioester or selenoester compounds may, in certain embodiments, comprise the formula:

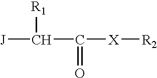

wherein J comprises a residue of the organic backbone; R₁ comprises any side chain group; X is sulfur or selenium; and R₂ is any thioester or selenoester compatible group; and wherein one or more of R₁ and R₂ is a group that sterically hinders the thioester or selenoester moiety —C(O)—X—. More specifically, one or more of R. and R₂ may comprise a branching group having the formula:

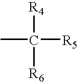

wherein R₄, R₅ and R₆ each individually are hydrogen or a linear, branched, substituted and unsubstituted alkyl, aryl, heteroaryl, and benzyl groups, with the proviso that two or more of R₄, R₅ and R₆ are linear, branched, substituted and unsubstituted alkyl, aryl, heteroaryl, and benzyl groups. The groups X and R₁-R₆ are the same as described above.

The methods for producing sterically hindered thioester or selenoester compounds may more specifically comprise: providing a composition of the formula:

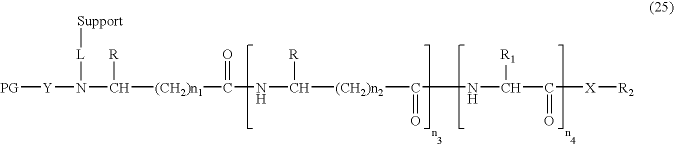

wherein PG, Y, L, R, R₁, R₂, X, n₁, n₂, n₃, n₄ and the Support are the same as described above for the structure (2); and cleaving the linker L under non-nucleophilic conditions to generate a sterically hindered thioester or selenoester compound free of the support. The sterically hindered thioester or selenoester compound thus freed from the support may have the formula:

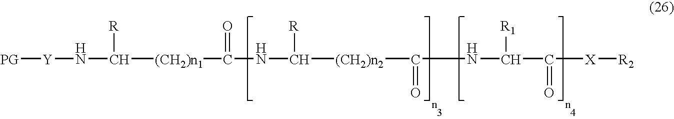

where PG is removable under conditions orthogonal to cleavage of linker L or, where PG is removable under the same conditions used for cleavage of linker L, the sterically hindered thioester or selenoester compound may comprise the formula:

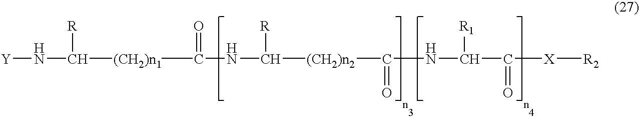

where the groups PG, Y, R, R₁, R₂, X, n₁, n₂, n₃, n₄ are as provided above. Here again, where R, R₁ and R₂ bear functional groups, each group R, R₁ and R₂ individually may be fully protected, or partially or totally unprotected following cleavage from the support, depending on the intended end use.

As described above, the invention can be used in nucleophile-based synthesis schemes. The invention finds particular use in the nucleophile-based synthesis of polyamide thioester and selenoester generators, and more particularly, peptide thioester and selenoester generators, and their associated intermediates and products. For instance, the O-alpha-carboxyl group of a backbone anchored amino acid or peptide is protected with a protecting group that is orthogonal to the nucleophile-labile group used in the SPPS chain assembly chemistry. With Fmoc-SPPS, for example, an allyl, ODmab, or photolytic group may be employed for protecting the C-terminal carboxylate. After SPPS chain-assembly of a selected polyamide is performed from the alpha-amino end of the anchored compound, the alpha-carboxyl group of the anchored compound is deprotected and activated. Then, a preformed amino acid or peptide -thioester or -selenoester derivative is acylated with the activated alpha-carboxyl group to provide C-terminal thioester or selenoester functionality usable for subsequent reactions once cleaved from the support, such as, for example, use of the product in chemical ligation reactions. Cleavage of the linker results in the generation of the target thioesters or selenoester product.

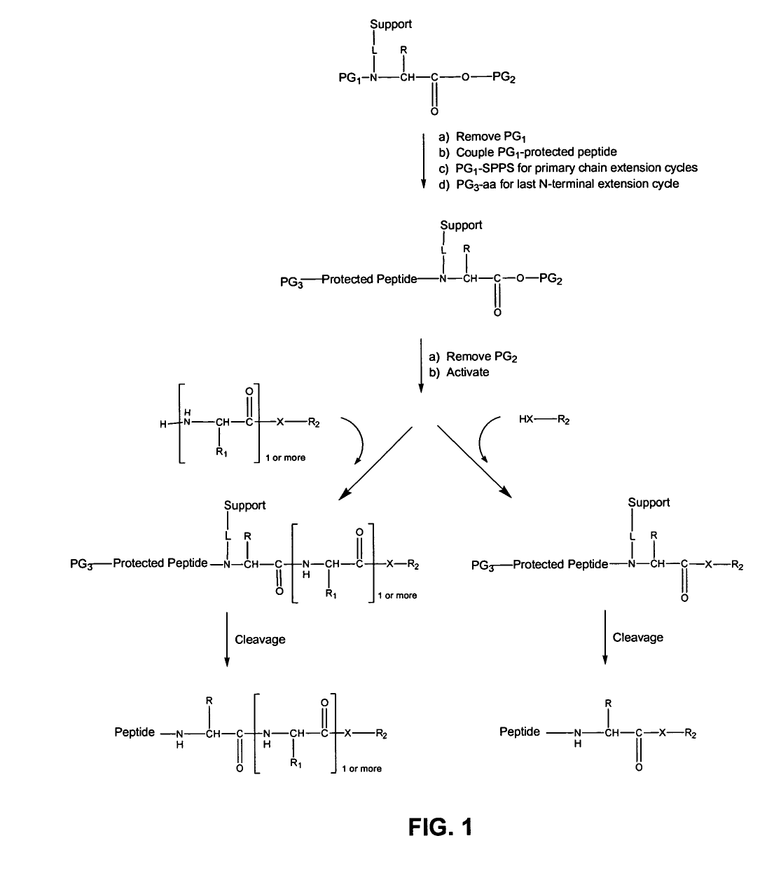

The invention will be more fully understood by reference to the reaction schemes shown in FIG. 1 through FIG. 4 with respect to preferred embodiments of compositions and methods.

Referring first to FIG. 1, an overview of a preferred method of production of thioester and selenoester generators and peptides in accordarice with the invention is illustrated. In the reaction scheme of FIG. 1, an amino acid synthon is provided that includes (i) an N-terminal group having a backbone nitrogen anchored to a support through a nucleophile-stable linker and protected with a nucleophile-labile protecting group PG1, (ii) a C-terminal carboxyl protected with a carboxyl protecting group PG2 that is removable under conditions orthogonal to PG₁, and (iii) R, which is either a hydrogen or an organic side chain that lacks reactive functional groups. In FIG. 1, the amino acid synthon is a single amino acid, although an amino acid synthon with more than one amino acid may also be used. As shown in FIG. 1, the amino acid synthon is extended by deprotecting the N-terminal group of the amino acid synthon and coupling a protected peptide of interest, preferably a dipeptide and more preferably a tripeptide or higher peptide, to the N-terminal group. This chain extension in the N- to C-terminal direction may be repeated stepwise with additional peptides or other target molecules of interest bearing the nucleophile-labile protecting group PG₁. Once the desired chain assembly is achieved, a pendant amino acid or peptide bearing a nucleophile-stable protecting group PG₃ (or, in the reaction scheme of FIG. 1, the PG3-protected peptide of interest) is coupled during the last N-terminal extension cycle. The carboxyl protecting group PG2 is selectively removed, generating a free carboxylate on the C-terminal end of the elongated amino acid synthon. The free carboxylate is then activated and reacted with a preformed thioester or selenoester, or a thiol or selenol bearing compound to form a thioester or selenoester generator, as depicted in FIG. 1, where X is sulfur or selenium, R₁ is either a hydrogen or any organic side chain group, and R₂ is any group compatible with thioesters or selenoesters. One or more than one of R, R₁ and R₂ may be a group that sterically hinders the thioester or selenoester moiety —C(O)—X— of the generator.

Still referring to FIG. 1, following the conversion of the extended amino acid synthon to the thioester or selenoester generator, the peptide of interest can be further modified while still bound to the support, or cleaved to release the desired thioester or selenoester peptide. As shown, the released peptide is deprotected. However, partially protected or even fully protected peptides can be made by employing protecting groups orthogonal to cleavage conditions.

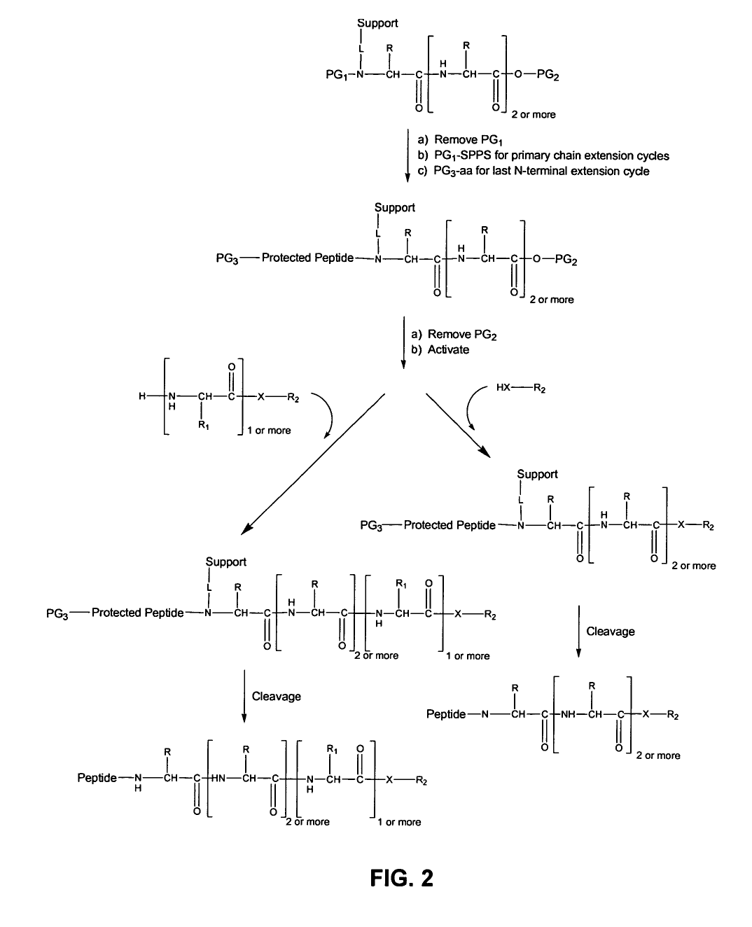

Turning now to FIG. 2, an overview of another preferred method of production of thioester and selenoester generators and peptides in accordance with the invention is illustrated. In FIG. 2, an amino acid synthon similar to that of FIG. 1 is provided, except the initial amino acid synthon of FIG. 2 includes at least three amino acid residues. The amino acid synthon is extended by a series of addition (deprotection/coupling) cycles that involve adding a Nα-PG₁-protected amino acid or peptide components stepwise in the N- to C-terminal direction. Once the desired chain assembly is achieved, a pendant amino acid or peptide bearing a nucleophile-stable protecting group PG₃ is coupled during the last N-terminal extension cycle. The carboxyl protecting group PG₂ is selectively removed, generating a free carboxylate on the C-terminal end of the elongated amino acid synthon. The free carboxylate is then activated and reacted with a preformed thioester or selenoester, or a thiol or selenol bearing compound to form a thioester or selenoester generator, as depicted in FIG. 2, where X is sulfur or selenium, R₁ is either a hydrogen or any organic side chain group, and R₂ is any group compatible with thioesters or selenoesters. One or more than one of R, R₁ and R₂ may be a group that sterically hinders the thioester or selenoester moiety —C(O)—X— of the generator. Similar to the reaction scheme of FIG. 1, following the conversion of the extended amino acid synthon to the thioester or selenoester generator, the peptide of interest can be further modified while still bound to the support, or cleaved to release the desired thioester or selenoester peptide. As shown, the released peptide is deprotected. However, partially protected or even fully protected peptides can be made by employing protecting groups orthogonal to cleavage conditions.

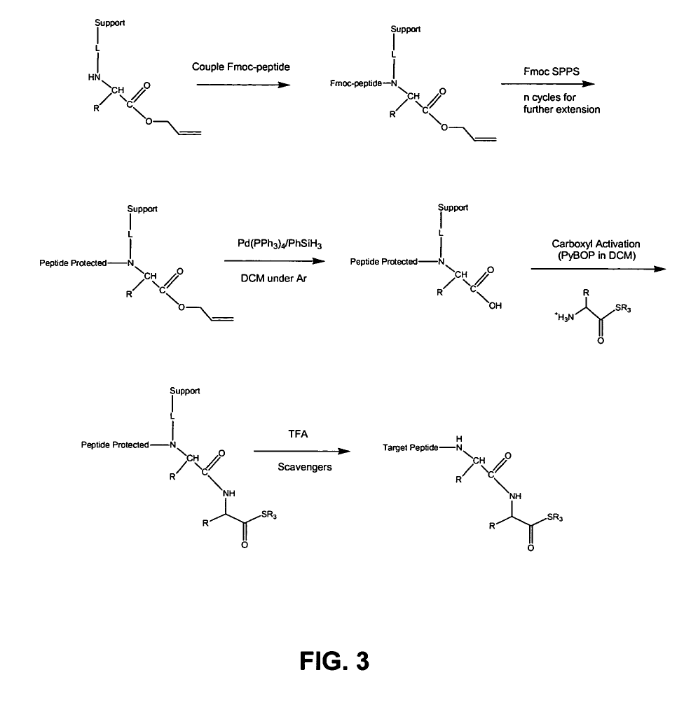

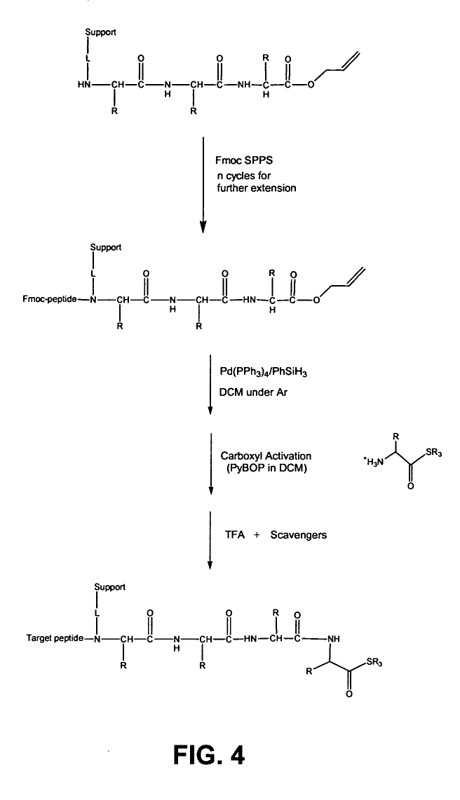

FIG. 3 and FIG. 4 illustrate reaction schemes specific to those discussed in Examples 2-5 and 7-11, respectively, and provide schematic references to the Examples.

As can be appreciated, the methods and compositions of the invention as described above, and exemplified in the Examples that follow have wide applicability in organic synthesis for the generation of thioesters and selenoesters. The subject compounds are particularly useful in peptide and polypeptide synthesis techniques that employ thioester and/or selenoester-mediated chemical ligation. Given the broad range of use, the subject thioester and selenoester generators and compounds also may be provided in kits and the like. The invention also allows for the production of activated thioesters and selenoesters from precursors that are prepared under strong nucleophilic conditions or non-nucleophilic synthesis schemes, or a combination of both. Thus, the invention has a wide range of uses and applications.

All publications and patent applications mentioned in this specification are herein incorporated by reference to the same extent as if each individual publication or patent application was specifically and individually indicated to be incorporated by reference.

Having now generally described the invention, the same will be more readily understood through reference to the following examples, which are provided by way of illustration, and are not intended to be limiting of the present invention, unless specified.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used but some experimental errors and deviations should be accounted for. Unless indicated otherwise, parts are parts by weight, molecular weight is weight average molecular weight, temperature is in degrees Centigrade, and pressure is at or near atmospheric.

### Example 1

General Materials

All reagents used are of the highest commercially available quality and are used as received. N^{α}- and side-chain protected Fmoc amino acids are obtained from Novabiochem, Bachem, Neosystems, and Fluka. Trifluoroacetic acid is obtained from Halocarbon Products. Solvents are HPLC grade. N,N-Dimethylformamide, acetonitrile, and water for HPLC purifications are from Burdick and Jackson. Water for buffer preparation is obtained from NERL Diagnostics.

Solid-Phase Peptide Synthesis

Peptides are synthesized in a stepwise manner on an ABI433 peptide synthesizer (Applied Biosystems, Foster City, Calif.) by SPPS using HBTU/DIEA/DMF coupling protocols at 0.1 mmol equivalent resin scale. For each standard coupling cycle, 1 mmol N^{α}-Fmoc-amino acid, 4 mmol DIEA and 1 mmol equivalents of HBTU are used. The concentration of the HBTU-activated Fmoc amino acids is 0.5 M in DMF, and the couple time is 10 min. Fmoc deprotections are carried out with two treatments using a 30% piperidine in DMF solution for 2 minutes and then 18 minutes, followed by thorough DMF flow washes.

Analytical Methods

The crude and purified products are analyzed by reversed-phase HPLC on an Agilent 1100 instrument. The components are separated on a Vydac C₁₈ column using a 0-80% gradient of buffer B (90% acetonitrile, 9.92% water, 0.08% TFA) over buffer A (0.1% aqueous TFA) in 80 minutes.

Mass spectra are acquired on a PE-Sciex API-III triple-quadrupole mass spectrometer. Samples (10 μL) dissolved in acetonitrile/water are injected using a syringe pump (Harvard Apparatus) directly to the ionization source via a fused silica capillary interface (50 μm i.d.×30 cm length). Sample droplets are ionized at a positive potential of 5 kV and entered the analyzer through an interface plate and subsequently through an orifice (100-120 μm diameter) at a potential of 70 V. Full scan mass spectra are acquired over the mass range of 200-2000 Da with a scan step size of 0.1 Da. Molecular masses are derived from the observed m/z values using the MacSpec 3.3 software package (PE-Sciex, Toronto, Canada). Calculated theoretical monoisotopic and average masses are determined using the MacBiospec program (PE-Sciex Toronto, Canada).

### Example 2

(Schematic Illustration is Pprovided in FIG. 3)

Synthesis of BAL-resin

4-hydroxy-2,6-dimethoxybenzaldehyde (3.64 g, 20 mmol) is dissolved in anhydrous DMF (200 mL) in a three neck round bottom flask equipped with a mechanical stirrer and then purged with argon for 20 min. Sodium hydride (60% dispersion in mineral oil; ˜19 mmol) is slowly added portion-wise over 30 mins. Merrifield resin (5 mmol, loading 0.8mmol/g) is then added and stirred for 2 days at 50° C. The DMF suspension is then slowly diluted with isopropanol, methanol and filtered. The resin is washed with DMF/MeOH (50 mL), DMF (50 mL), DCM (50 mL), and ether (50 mL). The resin is then dried in vacuo for 3 days. Fourier-transformed infrared (FT-IR) is used to qualitatively confirm the presence of the expected aldehyde carbonyl stretch (˜1690 cm⁻¹).

Preparation of Glycine Allyl Ester, Trifluoroacetate Salt (H-Gly-OAllyl TFA)

Boc-Gly-OH (4.38 g, 25 mmol) is suspended in DMF (100 mL) and a solution of K₂CO₃ (6.91 g, 50 mmol) is added. After 10 minutes, allyl bromide (3.025 g, 25 mmol) is added and stirred at room temperature for 20 h. The DMF is removed by co-evaporation with toluene, and the residue is suspended in EtOAc (200 mL) and extracted with 10% aqueous NaHCO₃ (3×100 mL). The combined organic phases are concentrated in vacuo to an oil which is passed through a silica plug using EtOAc-hexane (1:4) as the eluent. The resultant oil is treated with 1:1 TFA-DCM for 15 minutes to remove the Boc group and then concentrated in vacuo to give an oil, and is then lyophilized twice after dilution with 1:1 acetonitrile/water to give the target compound.

Synthesis of H-(BAL-resin)Gly-0-allyl resin

Glycine 0-allyl ester, trifluoroacetate salt (4 mmol) is dissolved in 1% AcOH in DMF (2OmL) and added to dried BAL-resin (1 g, ˜0.8 mmol equiv) and left for 2 minutes. Sodium triacetoxyborohydride (1 g, 4.7mmol) is added and the reaction mixture is stirred thoroughly for 60 minutes, and at this time FT-IR analysis is used to monitor the disappearance of aldehyde carbonyl stretch. The resin is then washed with MeOH (3×30 mL), DMF (3×30 mL), DCM (2×30 mL), and methanol (2×30 mL). The H-(BAL-resin)Gly-O-allyl resin is then dried in vacuo.

### Example 3

(Schematic Illustration is Provided in FIG. 3)

Synthesis of Fmoc-Thr(O^{t}Bu)-Ser(O^{t}Bu)—OH

Dried Fmoc-Thr(OtBu)—OH (10 mmol; 3.975 g) is dissolved in DCM (50 mL) and cooled to 10° C. Dicyclohexylcarbodiimide (DCC, 1.03 g, 5 mmol) is then added and a white precipitate of N,N-dicyclohexylurea (DCU) formed within 1 minute and the reaction is left for 30 minutes. The DCU precipitate is separated from the supernatant by centrifugation and the supernatant is concentrated in vacuo. The resulting residue is then taken up in DMF (20 mL) and added to a freshly prepared solution containing NH₂-Ser(O^{t}Bu)-OAllyl (5 mmol), DIEA (10 mmol) and DMF (20 mL) and stirred for 5 hours. The reaction is monitored by TLC and once finished concentrated in vacuo with co-evaporation with toluene (3×50 mL) and dissolved in ethyl acetate (100 mL). The organic layer is then washed five times with 10% NaHCO₃, three times with 0.25M KHSO₄, five times with brine, and then water. The organic layer is then collected, dried over Na₂SO₄ for 20 minutes, and concentrated in vacuo. The C-terminal allyl ester is removed by treatment with Pd(PPh₃)₄ (25 mg; tetrakis(triphenylphosphine)palladium(0)) DCM in presence of phenylsilane (l00 mL; 1.05 mmol) with continuous argon purging at 25 OC for 1 hour. The reaction is then concentrated in vacuo and dissolved in ethyl acetate (100 mL). The organic layer is then washed two times with 0.25M KHSO4 and five times with brine. The organic layer is then collected, dried over Na₂SO₄ for 1 hour, and concentrated in vacuo. Crude Fmoc-Thr(OtBu)-Ser(OtBu)—OH is then purified by flash chromatography using a ethyl acetate/petroleum ether/acetic acid gradient solvent system.

Synthesis of RANTES(1-32)[(^{α}N-BAL Resin)Gly₃₂-O-Allyl

The SPYSS DTTPC CFAYI ARPLP RAHIK EYFYT S[(^{α}N-BAL Resin)Gly₃₂-O-allyl peptide is synthesized using the strategy described below and with the following side-chain and N-terminal protection strategy: Aspartic acid(O^{t}Bu), Arginine(Pbf), Cysteine(Acm), Glutamic acid(O^{t}Bu), Glutamine(Trt), Histidine(Trt), Lysine(N^{ε}-Boc), Serine(O^{t}Bu), Threonine(O^{t}Bu), Tyrosine(O^{t}Bu) and N^{α}-terminal Boc protection (ie. serine 1 is introduced by coupling with Boc-Ser(O^{t}Bu)—OH. The first segment of the peptide is coupled as a dipeptide to avoid diketopiperazine (DKP) formation that would be quite extensive especially with H-(BAL-resin)Gly-O-allyl resin when using conventional stepwise SPPS. To overcome this problem, the corresponding dipeptide Fmoc-Thr(OtBu)-Ser(OtBu)—OH (1 mmol) is coupled to (0.1 mmol) H-(BAL-resin)Gly-O-allyl resin using PyBOP (1 mmol) and collidine (5 mmol) in DCM:DMF (1:1) at a concentration of 0.1 M. For each coupling cycle following this, 1 mmol N^{α}-Fmoc-amino acid, 4 mmol DIEA and 1 mmol equivalents of HBTU are used. The concentration of the HBTU-activated Fmoc amino acids is 0.5 M in DMF, and the couple time is 10 minutes. Fmoc deprotections are carried out with two treatments using a 30% (v/v) piperidine in DMF solution for 2 min and then 18 minutes.

### Example 4

(Schematic Illustration is Provided in FIG. 3)

Allyl deprotection of RANTES(1-32)[^{α}N-BAL Resin)Gly₃₂O-AIlyl

0.1 mmol of RANTES(1-32)[(^{α}N-BAL Resin)GIy₃₂-O-Allyl is swollen in dry DCM for 1 h. The C-terminal allyl ester is removed by two treatments with Pd(PPh₃)₄ (25 mg; tetrakis(triphenylphosphine)palladium(0)) DCM in presence of phenylsilane (100 μL; 1.05 mmol) with continuous argon purging at 25° C. for 30min. The RANTES(1-32)[(^{α}N-BAL Resin)Gly₃₂-OH resin is then washed with degassed DCM, DMF, DMF/MeOH, and DCM, and dried in vacuo for 3 hours.

Synthesis of RANTES(1-31)[(^{α}N-BAL Resin) Gly₃₂-Lys₃₃-^{α}COS-CH₂CH₂COOEt

0.1 mmol of RANTES(1-32)[(^{α}N-BAL Resin)Gly₃₂-OH resin is swollen in anhydrous DCM (1 mL) for 10 minutes. NH2-Lys(N^{ε}-Boc)-^{α}COS-CH₂CH₂-COOEt (10 mmol) in DMF (2 mL) and DIEA (3 mmol) added, and the reaction is mixed thoroughly. Solid PyBOP (or DIC) (1 mmol) is then immediately added directly to the resin-mixture, mixed thoroughly and left for 1 hour. This coupling procedure is repeated. The resin is then drained, and washed with DCM, DMF, DCM, and then dried in vacuo for 1 hour. This procedure is repeated. The RANTES(1-32)[(N^{α}-BAL Resin)Gly₃₂-Lys₃₃-^{α}COS-CH₂CH₂COOEt resin is then drained, and washed with DCM, DMF, DCM, and then dried in vacuo for 1 hour.

### Example 5

(Schematic illustration is provided in FIG. 3)

TFA Cleavage of RANTES(1-32)[(^{α}N-BAL Resin)Gly₃₂Lys₃₃-^{α}COS-CH₂CH₂COOEt

The peptide-resin is deprotected and released by treatment with a TFA/TIS/H₂O (95:2.5:2.5, v/v) solution at room temperature for 1 hour. The volatiles are then removed with a stream of nitrogen over 10 minutes, precipitated twice with diethyl ether and separated by centrifugation, and the product is extracted with 50% acetonitrile/water. The resin is filtered off and the aqueous solution containing RANTES(1-32)[Gly₃₂-Lys₃₃-^{α}COS-CH₂CH₂COOEt is lyophilized. The RANTES(1-32)[Gly₃₂-Lyd₃₃-^{α}COS-CH₂CH₂COOEt peptide is then purified by preparative RP-HPLC.

### Example 6

Ligation of RANTES(1-32)[Lys₃₃-^{α}COS-CH₂CH₂COOEt] with RANTES(34-68)

The RANTES(1-32)[Lys₃₃-^{α}COS-CH₂CH₂COOEt] thioester peptide prepared in Example 5 is ligated to the corresponding N-terminal cysteine peptide of RANTES, RANTES(33-68), CSNPAWFVTRKNRQVCANPEKKWVREYINSLEMS. Approximately 10 mg of the RANTES(1-32)[Lys₃₃-^{α}COS-CH₂CH₂COOEt] thioester peptide and 8 mg of RANTES(34-68) peptide are transferred into an Eppendorf tube and dissolved in 0.2 mL of a 6M Gn.HCl, 300 mM phosphate buffer at pH 7.0 solution containing 1% thiophenol. The ligation reaction is allowed to proceed for 18 hours at room temperature. The reaction is worked-up by the addition of P3-mercaptoethanol (0.05mL) and TCEP (10 mg), quenched by the addition of 0.5 mL 6M Gn.HCl, 100 mM acetate buffer at pH 4 and then analyzed by analytical RP-HPLC and electrospray mass spectrometry. The target product, RANTES(1-68), is purified by preparative RP-HPLC.

### Example 7

(Schematic illustration is provided in FIG. 4)

Synthesis N-Fmoc-(BAL-resin) Thr(O^{t}Bu)-Ser(O^{t}Bu)-Gly-O-allyl resin

Threonine(O^{t}Bu)-O-allyl ester (4 mmol) is dissolved in 1% AcOH in DMF (20 mL) and added to dried BAL-resin (1 g, ˜0.8 mmol equiv) and left for 2 minutes. Sodium triacetoxyborohydride (1 g, 4.7mmol) is added and the reaction mixture is stirred thoroughly for 60 minutes, and at this time FT-IR analysis is used to monitor the disappearance of aldehyde carbonyl stretch. The resin is then washed with MeOH (3×30 mL), DMF (3×30 mL), DCM (2×30 mL), and methanol (2×30 mL). The H-(BAL-resin)Thr(O^{t}Bu)-O-allyl resin is then dried in vacuo. The resin is then swollen in DMF and neutralized with 10% DIEA treatments and wash thoroughly with DMF. The N^{α}-amino group is then Fmoc-protected by two treatments with 0.2 M Fmoc-OSu (10mmol) in DMF for 2 hours. N^{α}-Fmoc-(BAL-resin)Thr(O^{t}Bu)-O-allyl ester is then washed thoroughly with DMF and DCM. The C-terminal allyl ester is removed by two treatments with Pd(PPh₃)₄ (25 mg; tetrakis(triphenylphosphine)palladium(0)) DCM in presence of phenylsilane (100 μL; 1.05 mmol) with continuous argon purging at 25° C. for 30 minutes. The N^{α}-Fmoc-(BAL-resin)Thr(O^{t}Bu)—OH resin is then washed with degassed DCM, DMF, DMF/MeOH, and DCM, and dried in vacuo for 3 hours. The N^{α}-Fmoc-(BAL-resin)Thr(O^{t}Bu)—OH resin is swollen in DMF and the dipeptide, NH₂-Ser(O^{t}Bu)-Gly-O-allyl ester (4 mmol), is added with collidine (10 mmol). After thoroughly mixing, the resin mixture, solid PyAOP (or DIC) 2 mmol is added, mixed and left for 2 hours. The N^{α}-Fmoc-(BAL-resin)Thr(O^{t}Bu)-Ser(O^{t}Bu)-Gly-O-allyl resin is then washed with DCM, DMF, and DCM, and dried in vacuo.

### Example 8

(Schematic Illustration is Provided in FIG. 4)

Synthesis of RANTES(1-29)[(N^{α}-BAL Resin)Thr₃o(O^{t}Bu)-Ser₃l(O^{t}Bu)-GIy₃₂-O-allyl] resin

The SPYSS DTTPC CFAYI ARPLP RAHIK EYFY [(N^{α}-BAL Resin)Thr₃₀(O^{t}Bu)-Ser₃₁(O^{t}Bu)-Gly₃₂-O-allyl] resin peptide is synthesized using the strategy described below and with the following side-chain and N-terminal protection strategy: Aspartic acid(O^{t}Bu), Arginine(Pbf, Cysteine(Acm), Glutamic acid(O^{t}Bu), Glutamine(Trt), Histidine(Trt), Lysine(N^{ε}-Boc), Serine(O^{t}Bu), Threonine(O^{t}Bu), Tyrosine(O^{t}Bu) and N^{α}-terminal Boc protection (i.e., serine 1 is introduced by coupling with Boc-Ser(O^{t}Bu)—OH). The concentration of the activated HBTU-activated Fmoc amino acids is 0.5 M in DMF, and the couple time is 10 minutes. Fmoc deprotections is carried out with two treatments using a 30% (v/v) piperidine in DMF solution for 2 minutes and then 18 minutes.

### Example 9

(Schematic Illustration is Provided in FIG. 4)

Allyl deprotection of RANTES(1-29)[(N^{α}-BAL Resin)Thr₃₀(O^{t}Bu)-Ser₃l(O^{t}Bu)-Gly₃₂-O-allyl resin

0.1 mmol of RANTES(1-29)[(N^{α}-BAL Resin)Thr₃₀(O^{t}Bu)-Ser₃l(O^{t}Bu)-Gly₃₂-O-allyl resin is swollen in dry DCM for 1 hour. The C-terminal allyl ester is removed by two treatments with Pd(PPh₃)₄ (25 mg; tetrakis(triphenylphosphine)palladium(0)) DCM in presence of phenylsilane (100 μL; 1.05 mmol) with continuous argon purging at 25° C. for 30 minutes. The RANTES(1-29)[(N^{α}-BAL Resin)Thr₃₀(O^{t}Bu)-Ser₃₁(O^{t}Bu)-Gly₃₂-OH resin is then washed with degassed DCM, DMF, DMF/MeOH, and DCM, and dried in vacuo for 3 hours.

### Example 10

(Schematic Illustration is Provided in FIG. 4)

Synthesis of RANTES(1-29)[(N^{α}-BAL Resin)Thr30(O^{t}Bu)-Ser31(O^{t}Bu)-GIY32-Lys33(N^{ε}-Boc)-^{α}COS-CH₂CH₂COOEt resin

0.1 mmol of RANTES(1-29)[(N^{α}-BAL Resin) Thr₃₀(O^{t}Bu)-Ser₃₁(O^{t}Bu)-Gly₃₂-OH resin is swollen in anhydrous DCM (1 mL) for 10 minutes. NH2-Lys(N^{ε}-Boc)-^{α}COS-CH₂CH₂-COOEt (10 mmol) in DMF (2 mL) and DIEA (3 mmol) added, and the reaction mixed thoroughly. Solid PyBOP (or DIC) (1 mmol) is then immediately added directly to the resin-mixture, mixed thoroughly and left for 1 hour. This coupling procedure is repeated. The resin is then drained, and washed with DCM, DMF, DCM, and then dried in vacuo for 1 hour. This procedure is repeated. The RANTES(1-29)[(N^{α}-BAL Resin) Thr₃₀(O^{t}Bu)-Ser₃₁(tBu)-Gly₃₂-Lys₃₃(N^{ε}-Boc)-^{α}COS-CH₂CH₂COOEt resin is then drained, and washed with DCM, DMF, DCM, and then dried in vacuo for 1 hour.

### Example 11

(Schematic Illustration is Provided in FIG. 4)

TFA Cleavage of RANTES(1-29)[(N^{α}-BAL Resin) Thr₃₀(O^{t}Bu)-Ser₃₁(O^{t}Bu)-Gly₃₂-Lys₃₃(N^{ε}-Boc)-^{α}COS-CH₂CH₂COOEt resin

The RANTES(L -29)[(N^{α}-BAL Resin)Thr₃₀(O^{t}Bu)-Ser₃₁(O^{t}Bu)-Gly₃₂-Lys₃₃(N^{ε}-Boc)-^{α}COS-CH₂CH₂COOEt peptide-resin is deprotected and released by treatment with a TFA/TIS/H₂O (95:2.5:2.5) solution at room temperature for 1 hour. The volatiles are then removed with a stream of nitrogen over 10 minutes, precipitated twice with diethyl ether and separated by centrifugation, and the product extracted with 50% acetonitrile/water. The resin is filtered off and the aqueous solution containing RANTES(N^{ε}-32)[Lys₃₃(N^{ε}-Boc)-^{α}COS-CH₂CH₂COOEt] lyophilized. The RANTES(1-32)[Lys₃₃(N^{ε}-Boc)-^{α}COS-CH₂CH₂COOEt] peptide is then purified by preparative RP-HPLC.

### Example 12

Ligation of RANTES(1-32)[Lys₃₃-COS-CH₂CH₂COOEt] with RANTES(34-68)

The RANTES(1-32)[Lys₃₃-COS-CH₂CH₂COOEt] peptide prepared in Example 5 is ligated to the corresponding N-terminal cysteine peptide of RANTES, RANTES(33-68), CSNPAWFVTRKNRQVCANPEKKWVREYINSLEMS. Approximately 10 mg of the RANTES(1-32)[Lys₃₃-COS-CH₂CH₂COOEt] thioester peptide and 8 mg of RANTES(34-68) peptide are transferred into an Eppendorf tube and dissolved in 0.2 mL of a 6M Gn.HCl, 3OOmM phosphate buffer at pH 7.0 solution with 1% thiophenol. The ligation reaction is allowed to proceed for 18 hours at room temperature. The reaction is worked-up by the addition of β-mercaptoethanol (0.05mL) and TCEP (10 mg), quenched by the addition of 0.5 mL 6M Gn.HCl, 100 mM acetate buffer at pH 4 and then analyzed by analytical RP-HPLC and electrospray mass spectrometry. The target product, RANTES(1-68), is then purified by preparative RP-HPLC.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A thioester or selenoester generator comprising an amino acid synthon having an N-terminal group joined to a C-terminal group through an organic backbone having side chains lacking reactive functional groups, said N-terminal group comprising a first amino acid residue having a backbone nitrogen anchored to a support through a nucleophile-stable linker, and said C-terminal group comprising a second amino acid residue having a backbone carbonyl of an ester, wherein said first and second amino acid residues are separated by one or more additional amino acid residues.

2. The thioester or selenoester generator according to claim 1, wherein said backbone nitrogen is protected with an amino protecting group.

3. The thioester or selenoester generator according to claim 1, wherein said backbone nitrogen is coupled to an additional amino acid or peptide.

4. The thioester or selenoester generator according to claim 3, wherein said additional amino acid or peptide is capable of supporting chemical ligation.

5. The thioester or selenoester generator according to claim 4, wherein said additional amino acid or peptide comprises a side chain group.

6. The thioester or selenoester generator according to claim 4, wherein said additional amino acid or peptide comprises a terminal group.

7. The thioester or selenoester generator according to claim 1, wherein said ester is a member selected from the group consisting of a thioester and a selenoester.

8. A thioester or selenoester generator comprising the formula: 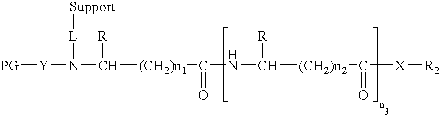
wherein:
Y is a target molecule of interest that may be present or absent, and is lacking reactive functional groups;
PG is a protecting group that may be present or absent, and is an amino protecting group when Y is absent;
L is a nucleophile-stable linker;
Support is chosen from a solid phase, matrix, or surface;
R is hydrogen or any organic side chain lacking reactive functional groups;
n₁ and n₂ each are from 0 to 2;
n₃ is from 2 to 20;
X is oxygen, sulfur, or selenium;
R₂ is a protecting group removable under conditions orthogonal to PG when X is oxygen; and
R₂ is any group compatible with thioesters or selenoesters when X is sulfur or selenium.

9. A method of generating a thioester or selenoester, said method comprising:
(a) providing a composition comprising an amino acid synthon having an N-terminal group joined to a C-terminal group through an organic backbone lacking reactive functional groups, said N-terminal group comprising a first amino acid residue having a backbone nitrogen anchored to a support through a nucleophile-stable linker, and said C-terminal group comprising a second amino acid residue having a backbone carboxyl protected with a carboxyl protecting group removable under conditions orthogonal to said nucleophile-stable linker, wherein said first and second amino acid residues are separated by one or more additional amino acid residues;
(b) forming an elongated product having one or more additional amino acids or peptides that extend from, and is covalently joined to, said backbone nitrogen, with the proviso that said elongated product is lacking reactive functional groups;
(c) selectively removing said carboxyl protecting group from the product of step (b) to generate a free carboxylate; and
(d) converting said free carboxylate to a thioester or selenoester to generate said thioester or selenoester.

10. The method according to claim 9, further comprising cleaving said nucleophile-stable linker under non-nucleophilic conditions.

11. A method of producing a thioester or selenoester generator, said method comprising:
(a) providing a composition comprising an amino acid synthon having an N-terminal group joined to a C-terminal group through an organic backbone having side chains lacking reactive functional groups, said N-terminal group comprising a first amino acid residue having a backbone nitrogen anchored to a support through a nucleophile-stable linker and lacking reactive functional groups, and said C-terminal group comprising a second amino acid residue having a free backbone carboxyl, wherein said first and second amino acid residues are separated by one or more additional amino acid residues; and
(b) converting said backbone carboxyl to a thioester or selenoester.

12. The method according to claim 11, further comprising cleaving said nucleophile-stable linker under non-nucleophilic conditions.

13. A method of producing a thioester or selenoester generator, comprising:
(a) providing a precursor compound having the formula: 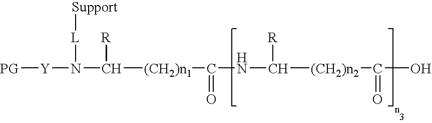
wherein:
Y is a target molecule of interest that may be present or absent, and is lacking reactive functional groups;
PG is a protecting group that may be present or absent, and is an amino protecting group when Y is absent;
L is a nucleophile-stable linker;
Support is chosen from a solid phase, matrix, or surface;
R is hydrogen or any organic side chain lacking reactive functional groups;
n₁ and n₂ each are from 0 to 2; and
n₃ is from 2 to 20; and
(b) converting said C-terminal carboxyl of said precursor compound to a thioester or selenoester.

14. The method according to claim 13, further comprising cleaving said nucleophile-stable linker under non-nucleophilic conditions.

15. A method of generating a thioester or selenoester, comprising:
(a) providing a composition comprising an amino acid synthon having an N-terminal group joined to a C-terminal group through an organic backbone lacking reactive functional groups, said N-terminal group comprising an unprotected backbone nitrogen anchored to a support through a nucleophile-stable linker, and said C-terminal group comprising a backbone carboxyl protected with a carboxyl protecting group that is removable under conditions orthogonal to said nucleophile-stable linker;
(b) coupling a peptide to said unprotected backbone nitrogen, said peptide having a C-terminal group comprising an activated carboxyester and an N-terminal group comprising an amino group protected with an amino protecting group removable under conditions orthogonal to said carboxyl protecting group;
(c) optionally, selectively removing said amino protecting group from the product of step (b) to generate an unprotected amino group, and producing an elongated product having one or more amino acids or peptides that extend from, and are covalently joined to said unprotected amino group, with the proviso that said elongated product is lacking reactive functional groups;
(d) selectively removing said carboxyl protecting group from the product of step (b) or (c) to generate a free carboxyl group; and
(e) converting said free carboxyl group to a thioester or selenoester to produce said thioester or selenoester.

16. The method according to claim 15, further comprising cleaving said nucleophile-stable linker under non-nucleophilic conditions.

17. A method of generating a thioester or selenoester, said method comprising:
(a) providing:
(i) a precursor compound having the formula: 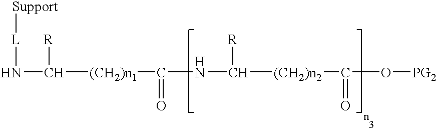
wherein:
L is a nucleophile-stable linker;
Support is chosen from a solid phase, matrix, or surface;
R is hydrogen or any organic side chain lacking reactive functional groups;
n₁ and n₂ each are from 0 to 2;
n₃ is from 0 to 20; and
PG₂ is a carboxyl protecting group that is removable under conditions orthogonal to L; and
(ii) a peptide of the formula: 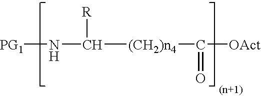
wherein:
PG₁ is an amino protecting group removable under conditions orthogonal to PG₂;
n4 is 0 to 2;
n is 1 to 20; and
OAct is an activated ester;
(b) coupling said peptide of step (a)(ii) to the unprotected amino group of the precursor compound of step (a)(i) to generate a composition having the formula: 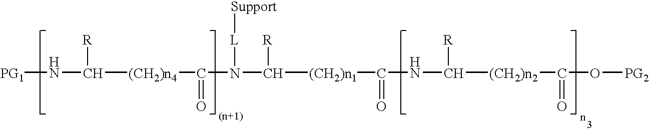
(c) optionally, selectively removing said amino protecting group PG₁ from the product of step (b) and forming an elongated product having the formula: 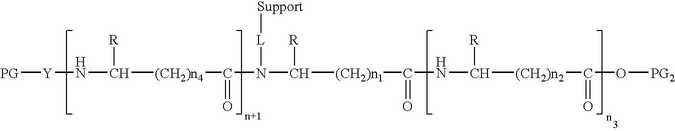
wherein:
Y is a target molecule of interest and is lacking reactive functional groups; and
PG is a protecting group that may be present or absent and is removable under conditions orthogonal to said carboxyl protecting group PG₂;
(d) selectively removing said carboxyl protecting group from the product of step (b) or (c) to generate a free carboxyl, and converting said free carboxyl to a thioester or selenoester to generate a thioester or selenoester generator of the formula: 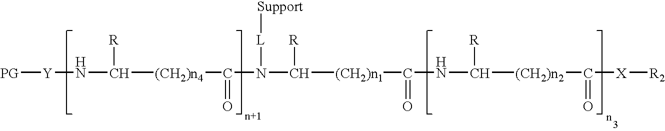
wherein:
PG and Y may be individually present or absent;
X is sulfur or selenium; and
R₂ is any group compatible with thioesters or selenoesters.

18. The method according to claim 17, further comprising cleaving said nucleophile-stable linker under non-nucleophilic conditions.

19. A sterically hindered thioester or selenoester generator comprising an amino acid synthon having an N-terminal group joined to a C-terminal group through an organic backbone, said N terminal group comprising a backbone nitrogen anchored to a support through a nucleophile-stable linker, and said C-terminal group comprising a moiety chosen from a sterically hindered thioester or selenoester.

20. The sterically hindered thioester or selenoester generator according to claim 19, wherein said amino acid synthon comprises two or more amino acid residues.

21. The sterically hindered thioester or selenoester generator according to claim 19, wherein said C-terminal group comprises a backbone carbonyl.

22. A sterically hindered thioester or selenoester generator comprising the formula: 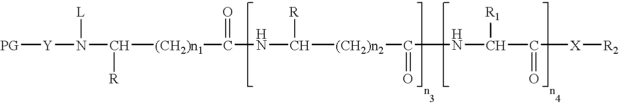
wherein:
Y is a target molecule of interest that may be present or absent, and is lacking reactive functional groups;
PG is a protecting group that may be present or absent, and is an amino protecting group when Y is absent;
L is a nucleophile-stable linker; each R and R₁ individually is hydrogen or any organic side chain group and may be the same or different;
n₁ and n₂ each individually are 0, 1 or 2;
n₃ is 0 to 20;
n₄ is 0 or 1;
X is sulfur or selenium;
R₂ is any group compatible with a thioester or selenoester; and
at least one of R, R₁ and R₂ is a group that sterically hinders the thioester or selenoester moiety —C(O)—X—.

23. A method of producing a sterically hindered thioester or selenoester generator, said method comprising:
(a) providing a precursor composition comprising an amino acid synthon having an N-terminal group joined to a C-terminal group through an organic backbone lacking reactive functional groups, said N-terminal group comprising a nitrogen anchored to a support through a nucleophile-stable linker, and said C-terminal group comprising a free carboxyl; and
(b) converting said free carboxyl of said precursor composition to a sterically hindered thioester or selenoester.

24. The method according to claim 23, further comprising cleaving said nucleophile-stable linker under non-nucleophilic conditions.

25. A method of producing a sterically hindered thioester or selenoester generator, said method comprising:
(a) providing a precursor composition having the formula: 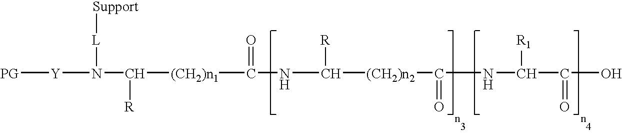
wherein:
Y is a target molecule of interest that may be present or absent, and is lacking reactive functional groups;
PG is a protecting group that may be present or absent, and is an amino protecting group when Y is absent;
Support is chosen from a solid phase, matrix, or surface;
L is a nucleophile-stable linker;
each R and R₁ individually is hydrogen or any organic side chain group and may be the same or different;
n₁ and n₂ each individually are 0, 1 or 2;
n₃ is 0 to 20; and
n₄ is 0 or 1; and
(b) converting the free carboxyl of the precursor composition step (a) to a sterically hindered thioester or selenoester to produce a sterically hindered thioester or selenoester having the formula: 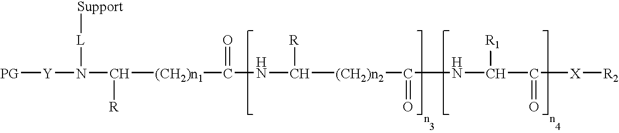
wherein:
X is sulfur or selenium;
R₂ is any thioester or selenoester compatible group; and
at least one of R, R, and R₂ is a group that sterically hinders the thioester or selenoester moiety —C(O)—X—.

26. The method according to claim 25, further comprising cleaving said nucleophile-stable linker under non-nucleophilic conditions.
